(19) United States
(12) Patent Application Publication
(10) Pub. No.: US 2011/0280827 A1
(43) Pub. Date: Nov. 17, 2011

(54) METHODS AND COMPOSITIONS FOR TREATING HEMATOLOGICAL MALIGNANCIES

(76) Inventors: Yanping Hu, Westborough, MA (US); William Siders, Franklin, MA (US); Johanne Kaplan, Sherborn, MA (US)

(21) Appl. No.: 13/146,247

(22) PCT Filed: Jan. 28, 2010
(86) PCT No.: PCT/US10/22420

§ 371 Date: Jul. 26, 2011

Related U.S. Application Data
(60) Provisional application No. 61/148,904, filed on Jan. 30, 2009.

(51) Int. Cl. A61K 38/19; A61P 35/00; A61P 35/02; A61K 39/395

(52) U.S. Cl. 424/85.1; 424/133.1

(57) ABSTRACT

The invention relates to methods and compositions for treating a subject afflicted with a hematological malignancy using a combination of a CXCR4 antagonist and an immunotherapeutic agent.

### TECHNICAL FIELD

This invention is in the field of treating hematological malignancies. In particular, the invention concerns methods and compositions for treating hematological malignancies using a combination of a CXCR4 antagonist and an immunotherapeutic agent.

### BACKGROUND ART

A common approach to treating a hematological malignancy is a session of immunotherapy to destroy the malignant cells combined with transplantation of hematopoietic progenitor cells either of autogeneic or allogeneic origin. Monoclonal antibodies are a widely used form of cancer immunotherapy at this time. Their side effects are relatively mild compared with the side effects of chemotherapy, mainly due to the higher specificity of tumor cell targeting. It is believed that the lack of success experienced with this treatment regimen is due to diminished ability of the immunotherapy to completely eliminate the malignant hematopoietic cells or their precursors. The present invention improves this method by combining immunotherapy with CXCR4 antagonists.

The compounds useful in invention are antagonists of the CXCR4 receptor that prevent its interaction with the cytokine stromal cell derived factor-1 (SDF-1), which is now designated as CXCL12. Many such agents and uses of such agents are known in the art. One notable agent is 1,1′-[1,4-phenylene-bis-(methylene)-bis-1,4,8,11-tetraazacyclotetradecane (also known by its codename, AMD3100), which is the active ingredient of MOZOBIL® (plerixafor), which is approved by the FDA for use in combination with granulocyte-colony stimulating factor (G-CSF) to mobilize hematopoietic stem cells to the peripheral blood for collection and subsequent autologous transplantation in patients with non-Hodgkin's lymphoma (NHL) and multiple myeloma (MM). This CXCR4 antagonist and other CXCR4 antagonists are disclosed, for example, in U.S. Pat. Nos. 5,021,409; 6,001,826; 5,583,131; 5,698,546; 5,817,807; 6,506,770; 6,756,391; 7,160,872; 6,872,714; 7,414,065; 6,667,320 and 7,022,717; in U.S. Patent Application Pub. Nos. 2007/0043012 and 2007/0060591; and in PCT Pub. Nos. WO 92/016494; WO 93/012096; WO 95/018808; WO 00/002870 and WO 01/044229, all of which are incorporated herein by reference.

The chemokine receptor CXCR4 and its natural ligand SDF-1/CXCL12 appear to be important in the process of hematopoiesis (for general reviews, see Maekawa, T., et al., Internal Med. (2000) 39:90-100; Nagasawa, T., et al., Int. J. Hematol. (2000) 72:408-411). For example, CXCR4 or SDF-1/CXCL12 knock-out mice exhibit hematopoietic defects (Ma, Q., et al., Proc. Natl. Acad. Sci USA (1998) 95:9448-9453). It appears that SDF-1/CXCL12 is able to control the positioning and differentiation of cells bearing CXCR4 receptors whether these cells are stem cells (i.e., cells which are CD34⁺) or are progenitor cells (which result in formation of specified types of colonies in response to particular stimuli).

It appears that, within the microenvironment of the bone marrow, SDF-1/CXCL12 acts as a potent chemoattractant for immature and mature hematopoietic cells, and thus expression of CXCR4 on leukemic progenitor cells and leukemia cells may contribute to homing them to the bone marrow microenvironment. Elevated CXCR4 levels are detected on leukemic cells from patients with B chronic lymphocytic leukemia (B-CLL) (Mohle, R., et al., Leukemia (1999) 13:1954-1959). It further appears that autocrine secretion of SDF-1/CXCL12 by blood-derived adherent nurse-like cells in chronic lymphocytic leukemia (CLL) protects leukemic B cells from spontaneous apoptosis (Burger, J. A., et al., Blood (2000) 96:2655-2663). Enhanced levels were not detected on leukemic cells from patients with T-ALL or leukemic cells from patients with AML according to Mohle, et al., supra; Voermans, C., et al., Leukemia (2002) 16:650-657; Bradstock, K. F., et al., Leukemia (2000) 14:882-888; Dialynas, D. P., et al., Stem Cells (2001) 19:443-452; Shen, W., et al., Exp. Hematol. (2001) 29:1439-1447. However, it appears that expression levels of CXCR4 vary among various types of AML as reported by Rombouts, E. J., et al., Blood (2004) 104:550-557; Fukuda, S., et al., Blood (2005) 105:3117-3126. CXCR4 was also reported to mediate homing and engraftment of pre-B-ALL and AML cells to bone marrow, although other factors may be involved (Shen, et al., supra; Tavor, S., et al., Cancer Res. (2004) 64:2817-2824).

It was shown, in an in vitro context, that AMD3100 blocked SDF-1/CXCL12 induced chemotaxis of pre-B-ALL cells into bone marrow stroma layers, and enhanced the cytotoxic and antiproliferative effects of vincristine and dexamethasone (Juarez, J., et al., Leukemia (2003) 17:1294-1300). Consistent with this result, another in vitro study found that AMD3465 and the polypeptide RCP168, both potent CXCR4 antagonists, significantly enhanced chemotherapy-induced apoptosis in stroma-cocultured Jurkat cells, primary CLL cells, and in a subset of AML cells harboring Flt3 mutation (Zeng, Z., et al., Mol. Cancer Ther. (2006) 5:3113-3121).

Recent in vivo studies in a murine model of AML found that administration of AMD3100 or AMD3465 to leukemic mice in combination with chemotherapy resulted in decreased tumor burden and improved overall survival compared to mice treated with chemotherapy alone (Nervi, B., et al., Blood, pre-published online Dec. 2, 2008, doi:10.1182/blood-2008-06-162123; Zeng, Z., et al., Blood, prepublished online Oct. 27, 2008, doi:10.1182/blood-2008-05-158311). A small in vivo human study has shown that the combination of AMD3100 and G-CSF produced massive mobilization of leukemic cells into the circulation in patients with AML who had insufficient mobilization of CD34+ cells with G-CSF alone (Andreeff, M., et al., Blood ASH Meeting Abstracts (2006) 108:Abstract 568). These studies indicate that SDF-1/CXCL12 and CXCR4 interactions are involved in the microenvironmental regulation of leukemic cells and such interaction plays a role in the resistance of residual, post-chemotherapy AML exposure to chemotherapeutic agents.

We have previously found, and have disclosed in PCT Pub. No. WO 00/045814, that the certain CXCR4 antagonists, such as AMD3100, have the effect of increasing the white blood cell count. We have also found, and have disclosed in PCT Pub. No. WO 03/011277, that these antagonists have the effect of mobilizing progenitor cells and/or stem cells from the bone marrow to the circulating blood. Certain uses of CXCR4 antagonists are disclosed in U.S. Patent Application Pub. Nos. 2007/0043012 and 2007/0060591; and PCT Pub. No. WO 08/019,371, all of which are incorporated herein by reference. U.S. Patent Application Pub. No. 2007/0043012, commonly assigned to the current applicant, discloses the use of CXCR4 antagonists to potentiate the effects of standard chemotherapeutic agents through the release and/or rapid movement of pre-leukemic cells and leukemic cells from the microenvironment of the bone marrow and into the circulating blood prior to, or during, or after treatment by chemotherapy. U.S. Patent Application Pub. No. 2007/0043012 does not specifically mention certain of the immunotherapeutic agents disclosed in the present invention.

There is currently a need for alternative or improved treatments of hematological malignancies. The current invention addresses such need by use of antagonists of the CXCR4 receptor in combination with immunotherapeutic agents, and it surprisingly has been found as demonstrated by the data presented herein that the combination of CXCR4 antagonists with an immunotherapeutic agent is of potential clinical significance. Moreover, we surprisingly have found that the dose of the CXCR4 antagonists is related to these findings insofar as a lower dose of the CXCR4 antagonist is associated with a greater potential therapeutic benefit.

Citation of the above documents is not intended as an admission that any of the foregoing is pertinent prior art. All statements as to the date or representation as to the contents of these documents is based on the information available to the applicants and does not constitute any admission as to the correctness of the dates or contents of these documents, and not intended to be bound by any theory or hypothesis described in these documents. Further, all documents referred to throughout this application are incorporated in their entirety by reference herein.

### DISCLOSURE OF THE INVENTION

In one aspect, the invention is directed to a method for treating a subject afflicted with a hematopoietic malignancy, which comprises administering a therapeutically effective amount of a CXCR4 antagonist as defined below in combination with an immunotherapeutic agent, such as, for example, a therapeutic antibody. The CXCR4 antagonist may be administered prior to, during, and/or after the immunotherapeutic regimen is administered.

In certain embodiments, the CXCR4 antagonist comprises a compound of the formula:

Z-linker-Z′ (1)
or a pharmaceutically acceptable salt or prodrug thereof,
wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;
Z′ may be embodied in a form as defined by Z above, or alternatively may be of the formula

—N(R)—(CR₂)ₙ—X
wherein each R is independently H or straight, branched or cyclic alkyl (1-6C), n is 1 or 2, and X is an aromatic ring, including heteroaromatic rings, or is a mercaptan,
or Z′ may be of the formula

—Ar(Y)ⱼ
wherein Ar is an aromatic or heteroaromatic moiety, and each Y is independently a non-interfering substituent and j is 0-3; and
“linker” represents a bond, alkylene (1-6C) or may comprise aryl, fused aryl, oxygen atoms contained in an alkylene chain, or may contain keto groups or nitrogen or sulfur atoms.

In certain embodiments, the CXCR4 antagonist comprises a compound of the formula:

Z-linker-Z′ (1)
or a pharmaceutically acceptable salt or prodrug thereof,
wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;
Z′ may be embodied in a form as defined by Z above, or alternatively may be of the formula

—N(R)—(CR₂)ₙ—X
wherein each R is independently H or straight, branched or cyclic alkyl (1-6C), n is 1 or 2, and X is an aromatic ring, including heteroaromatic rings, or is a mercaptan; and
“linker” represents a bond, alkylene (1-6C) or may comprise aryl, fused aryl, oxygen atoms contained in an alkylene chain, or may contain keto groups or nitrogen or sulfur atoms.

In another aspect, the invention is directed to a method for treating a subject afflicted with a hematopoietic malignancy, which comprises administering a therapeutically effective amount of a CXCR4 antagonist as defined below at a low dose. In certain embodiments, the low dose of a CXCR4 antagonist is administered in combination with an immunotherapeutic agent disclosed herein. In such combination therapy embodiments, the CXCR4 antagonist can be administered prior to, during, and/or after the immunotherapeutic regimen is administered.

In certain embodiments, the CXCR4 antagonist comprises a compound of the formula:

Z-linker-Z′ (1)
or a pharmaceutically acceptable salt or prodrug thereof,
wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;
Z′ may be embodied in a form as defined by Z above, or alternatively may be of the formula

—N(R)—(CR₂)ₙ—X
wherein each R is independently H or straight, branched or cyclic alkyl (1-6C), n is 1 or 2, and X is an aromatic ring, including heteroaromatic rings, or is a mercaptan,
“linker” represents a bond, alkylene (1-6C) or may comprise aryl, fused aryl, oxygen atoms contained in an alkylene chain, or may contain keto groups or nitrogen or sulfur atoms.

In another aspect, the invention is directed to a pharmaceutical or veterinary composition comprising a CXCR4 antagonist in unit dosage form for use in the methods of the invention. In certain embodiments, the composition comprises a CXCR4 antagonist and an immunotherapeutic agent and a suitable pharmaceutically or veterinary acceptable excipient. In certain embodiments, the CXCR4 antagonist comprises a compound of formula (1). In certain other embodiments, the CXCR4 antagonist is one disclosed herein.

Small molecule CXCR4 antagonists useful in the present invention are disclosed in U.S. Pat. Nos. 5,021,409; 6,001,826; 5,583,131; 5,698,546; 5,817,807; 6,506,770; 6,756,391; 7,160,872; 6,872,714; 7,414,065; 6,667,320 and 7,022,717; in U.S. Patent Application Pub. Nos. 2007/0043012 and 2007/0060591; and in PCT Pub. Nos. WO 92/016494; WO 93/012096; WO 95/018808; WO 00/002870 and WO 01/044229, all incorporated herein by reference.

In other embodiments, peptide-based antagonists may be used. These are described in PCT Pub. Nos. WO 01/85196; WO 00/09152 and WO 99/47158. The use of antibodies as antagonists of CXCR4 interacting with its ligand are disclosed in WO 99/50461. Other peptide-based compounds include T22 (Murakami, T., et al., J. Exp. Med. (1997) 186:1389-1393); T134 (Arakaki, R., et al., J. Virol. (1999) 73:1719-1723; T140 (Tamamura, H., et al., Biochem. Biophys. Res. Comm. (1998) 253:877-882) and its analogs TC14012 and TN14003 (Tamamura, H., et al., Bioorg. Med. Chem. Lett. (2001) 11:1897-1902; Mori, T., et al., Mol. Cancer Ther. (2004) 3:29-37; Burger, M., et al., Blood (2005) 106:1824-1830); ALX40-4C (Doranz, B. J., et al., J. Exp. Med. (1997) 186:1395-1400; Donzella, G. A., Nat. Med. (1998) 4:72-77; Doranz, B. J., et al., AIDS Res. Hum. Retrovir. (2001) 17:475-486); RCP168 (Zeng, Z., et al., Mol. Cancer Ther. (2006) 5:3113-3121); CTCE-0021 (Pelus, L. M., et al., Exp. Hematol. (2005) 33:295-307); CTCE-0214 (Zhong, R., et al., Exp. Hematol. (2004) 32:470-475); CTCE-9908 (Kim, S. Y., et al., AACR Meeting Abstracts (2005) Abstract 256); KRH-1120 (Yamamoto, N., et al., J. AIDS Res. (2000) 2:453-460); KRH-1636 (Ichiyama, K., et al., Proc. Natl. Acad. Sci. USA (2003) 100:4185-4190); KRH-2731 (Murakami T., et al., Abstracts of the 11th Conference on Retroviruses and Opportunistic Infections (2004) Abstract 541) and the like. As to the methods for preparation of these substances, they can be found, for example, in J. Exp. Med. (1997) 186:1189-1191 with any conventional modifications. All of these publications are incorporated herein by reference in their entireties.

### BRIEF DESCRIPTION OF THE DRAWINGS

FIG. 1 illustrates in vivo therapeutic efficacy of AMD3465 in severe combined immunodeficiency (SCID) mice bearing disseminated Raji lymphoma cells. AMD3465 significantly increased survival in the disseminated Raji tumor model at each concentration tested, with a stronger effect observed at the lower concentrations.

FIG. 2 illustrates in vivo therapeutic efficacy of AMD3465 in combination with CAMPATH® (alemtuzumab) in severe combined immunodeficiency (SCID) mice bearing disseminated Raji lymphoma cells. The combination of 5 mg/kg AMD3465 and CAMPATH® (alemtuzumab) significantly increased survival in the disseminated Raji tumor model compared to CAMPATH® (alemtuzumab) alone.

FIG. 3 illustrates in vivo therapeutic efficacy of AMD3100 in combination with RITUXAN® (rituximab) in severe combined immunodeficiency (SCID) mice bearing disseminated Raji lymphoma cells. The combination of 1.0 mg/kg AMD3100 and RITUXAN® (rituximab) significantly increased survival in the disseminated Raji tumor model compared to RITUXAN® (rituximab) alone.

FIG. 4 illustrates in vivo therapeutic efficacy of AMD3465 in combination with CAMPATH® (alemtuzumab) in severe combined immunodeficiency (SCID) mice bearing disseminated B104 lymphoma cells. The combination of AMD3465 and CAMPATH® (alemtuzumab) significantly increased survival in the disseminated B104 tumor model compared to CAMPATH® (alemtuzumab) alone at both concentrations of AMD3465 tested.

FIG. 5 shows another illustration of the in vivo therapeutic efficacy of AMD3465 in combination with CAMPATH® (alemtuzumab) in severe combined immunodeficiency (SCID) mice bearing disseminated B104 lymphoma cells. The combination of 5.0 mg/kg AMD3465 and CAMPATH® (alemtuzumab) significantly increased survival in the disseminated B104 tumor model compared to CAMPATH® (alemtuzumab) alone.

### MODES OF CARRYING OUT THE INVENTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted.

The discussion of the general methods given herein is intended for illustrative purposes only. Other alternative methods and embodiments will be apparent to those of skill in the art upon review of this disclosure.

As used herein, “a” or “an” means “at least one” or “one or more.”

A group of items linked with the conjunction “or” should not be read as requiring mutual exclusivity among that group, but rather should also be read as “and/or” unless expressly stated otherwise.

As used herein, the terms “treatment” or “treating” refers to any manner in which the symptoms of a condition, disorder or disease are ameliorated or otherwise beneficially altered. In the context of treating a hematological malignancy, the hematological malignancy can be onset, relapsed or refractory. Full eradication of the condition, disorder or disease is not required. Amelioration of symptoms of a particular disorder refers to any lessening of symptoms, whether permanent or temporary, that can be attributed to or associated with administration of a therapeutic composition of the present invention or the corresponding methods and combination therapies. Treatment also encompasses pharmaceutical use of the compositions in accordance with the methods disclosed herein.

As used herein, the term “subject” is not limited to a specific species or sample type. For example, the term “subject” may refer to a patient, and frequently a human patient. However, this term is not limited to humans and thus encompasses a variety of mammalian species.

The term “afflicted” as it relates to a disease or disorder refers to a subject having or directly affected by the designated disease or disorder.

As used herein, the term “hematological malignancy” refers to any type of cancer affecting blood cells, lymph nodes and/or bone marrow irrespective of whether such cancer is onset, relapsed or refractory. As used herein, the term “pre-malignant cells” refers to cells that can form malignant hematopoietic or myeloid cells. The malignant hematopoietic or myeloid cells are those which characterize the conditions of leukemia, lymphoma and myeloma. The three major categories of hematological malignancies are leukemia, lymphoma and myeloma. Leukemia is a cancer of the bone marrow and blood. The primary types of leukemia are lymphocytic leukemia, which involves an increase of white blood cells called lymphocytes, and myelogenous leukemia (also known as myeloid or myelocytic leukemia), which involves an increase in white blood cells called granulocytes. Leukemia can be acute or chronic. Acute forms of leukemia progress rapidly, whereas chronic forms of leukemia progress slowly, leading to different approaches to diagnosis and treatment. Examples of leukemia include acute lymphoblastic leukemia (ALL), acute myelogenous leukemia (AML), chronic myelogenous leukemia (CML), chronic lymphocytic leukemia (CLL), and hairy cell leukemia (HCL). Lymphoma is a general term for a group of cancers that originate in the lymph system. The two primary types of lymphoma are Hodgkin's lymphoma (HL), which spreads in an orderly manner from one group of lymph nodes to another; and non-Hodgkin's lymphoma (NHL), which spreads through the lymphatic system in a non-orderly manner. Myeloma, also known as multiple myeloma (MM), is a cancer of the plasma cells. In myeloma, the cells overgrow, forming a tumor located in the bone marrow. Bone marrow is the spongy tissue found in the center of the bone, where red cells, white blood cells, and platelets are produced.

As used herein, the terms “administration” or “administering” refers to any suitable method of providing a composition of the present invention to a subject. It is not intended that the present invention be limited to any particular mode of administration. In certain embodiments, the compounds and pharmaceutical compositions of the present invention are administered by a parenteral route, e.g., via intramuscular, intraperitoneal, intravenous, intracisternal or subcutaneous injection or infusion. The pharmaceutical compositions may be formulated in suitable dosage unit formulations appropriate for each route of administration.

As used herein, the term “effective amount” or “therapeutically effective amount” of a compound refers to a nontoxic but sufficient amount of the compound to provide the desired therapeutic or prophylactic effect to most patients or individuals. In the context of treating a hematological malignancy, a nontoxic amount does not necessarily mean that a toxic agent is not used, but rather means the administration of a tolerable and sufficient amount to provide the desired therapeutic or prophylactic effect to a patient or individual. The effective amount of a pharmacologically active compound may vary depending on the route of administration, as well as the age, weight, and sex of the individual to which the drug or pharmacologically active agent is administered. Those of skill in the art given the benefit of the present disclosure can easily determine appropriate effective amounts by taking into account metabolism, bioavailability, and other factors that affect plasma levels of a compound following administration within the unit dose ranges disclosed further herein for different routes of administration.

The term “immunotherapy” or “immunotherapeutic” generally refers to any therapeutic approach aimed at mobilizing or manipulating a patient's immune system to treat or cure disease. As used herein, the term “immunotherapy” or “immunotherapeutic” specifically refers to passive therapeutic strategies utilizing immune molecules such as antibodies as tumor-toxic agents or vehicles.

As used herein, the term “antibody” is used in the broadest sense, referring to monoclonal and polyclonal antibodies, whole antibodies, antibody fragments, and antibody sub-fragments that exhibit specific binding to a specific antigen of interest. Thus, “antibodies” can be whole immunoglobulin of any class, e.g., IgG, IgM, IgA, IgD, IgE. An “antibody” can be naturally occurring or man-made such as monoclonal antibodies produced by conventional hybridoma technology and/or a functional fragment thereof. Antibodies of the present invention comprise monoclonal and polyclonal antibodies as well as fragments containing the antigen-binding domain and/or one or more complementarity determining regions of these antibodies. The ability of a given molecule, including an antibody fragment or sub-fragment, to act like an antibody and specifically bind to a specific antigen can be determined by binding assays known in the art, for example, using the antigen of interest as the binding partner.

As used herein, the term “monoclonal antibody” or “mAb” refers to an antibody obtained from a population of substantially homogeneous antibodies, i.e., the antibodies comprising the population are identical except for possible naturally occurring mutations that are present in minor amounts. As used herein, a “monoclonal antibody” or “mAb” further refers to functional fragments of monoclonal antibodies. As used herein, a “monoclonal antibody” or “mAb” is not a species-specific term. For example, a “monoclonal antibody” may be a purely human antibody, a purely murine antibody, or a murine antibody in which some portions are replaced with human antibody fragments. Depending on how much of the mAb is human, the latter may be referred to as “chimeric” or “humanized” antibodies.

As noted above, in one aspect, the present invention is concerned with the use of a therapeutically effective amount of a CXCR4 antagonist in combination with an immunotherapeutic agent, such as, for example, a therapeutic antibody, to treat a subject afflicted with a hematological malignancy.

In another aspect, the invention is directed to a method for treating a subject afflicted with a hematopoietic malignancy, which comprises administering a therapeutically effective amount of a CXCR4 antagonist as defined below at a low dose. In certain embodiments, the low dose of a CXCR4 antagonist is administered in combination with an immunotherapeutic agent disclosed herein. In such combination therapy embodiments, the CXCR4 antagonist can be administered prior to, during, and/or after the immunotherapeutic regimen is administered.

In certain embodiments of the foregoing aspects, the CXCR4 antagonist is a compound of the formula:

Z-linker-Z′ (1)
wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;
Z′ may be embodied in a form as defined by Z above, or alternatively may be of the formula

—N(R)—(CR₂)ₙ—X
wherein each R is independently H or straight, branched or cyclic alkyl (1-6C),
n is 1 or 2, and
X is an aromatic ring, including heteroaromatic rings, or is a mercaptan;
or Z′ may be of the formula

—Ar(Y)ⱼ
wherein Ar is an aromatic or heteroaromatic moiety, and each Y is independently a non-interfering substituent and j is 0-3; and
“linker” represents a bond, alkylene (1-6C) or may comprise aryl, fused aryl, oxygen atoms contained in an alkylene chain, or may contain keto groups or nitrogen or sulfur atoms.

Specific forms of the compounds of formula (1) are discussed below.

In compounds of formula (1), certain embodiments of Z and Z′ are cyclic polyamine moieties having from 9-24C that include 3-5 nitrogen atoms, for example, 1,5,9,13-tetraazacyclohexadecane; 1,5,8,11,14-pentaazacyclohexadecane; 1,4,8,11-tetraazacylotetradecane; 1,4,7-triazacyclotetradecane; 1,5,9-triazacyclododecane; 1,4,7,10-tetraazacyclododecane; and the like, including such cyclic polyamines which are fused to an additional aromatic or heteroaromatic rings and/or containing a heteroatom other than nitrogen incorporated in the ring. Embodiments of Z and Z′ wherein the cyclic polyamine contains a fused additional cyclic system or one or more additional heteroatoms include, for example, 3,7,11,17-tetraazabicyclo(13.3.1)heptadeca-1(17),13,15-triene; 4,7,10,17-tetraazabicyclo[13.3.1]heptadeca-1(17),13,15-triene; 4,7,10-triazabicyclo[13.3.1]heptadeca-1(17),13,15-triene; and 4,10-diazabicyclo[13.31.1]heptadeca-1(17),13,15-triene. These and other related embodiments are described in U.S. Pat. No. 5,698,546 and PCT Pub No. WO 01/44229, incorporated herein by reference.

Embodiments of the linker moiety include those wherein the linker is a bond, or wherein the linker includes an aromatic moiety bracketed by two alkylene, preferably methylene moieties Linking groups include the methylene bracketed forms of 1,3-phenylene, 2,6-pyridine, 3,5-pyridine, 2,5-thiophene, 4,4′-(2,2′-bipyrimidine); 2,9-(1,10-phenanthroline) and the like. A particularly preferred linker is 1,4-phenylene-bis-(methylene).

In certain embodiments, the compounds include those of formula (1) wherein Z and Z′ are both cyclic polyamines. In certain other embodiments, Z and Z′ are identical. In further embodiments, Z is a cyclic polyamine that contains 10-24 members and contains 4 nitrogen atoms. In some specific embodiments, Z and Z′ are both 1,4,8,11-tetraazocyclotetradecane. Certain embodiments of the compound of the formula (1) include 2,2′-bicyclam and 6,6′-bicyclam; the embodiments set forth in U.S. Pat. Nos. 5,021,409, and 6,001,826, and in particular 1,1′-[1,4-phenylene-bis(methylene)]-bis-1,4,8,11-tetraazacyclotetradecane, set forth in U.S. Pat. No. 5,583,131, and sometimes designated herein as AMD3100.

When Z′ is other than a cyclic polyamine as defined in Z, certain embodiments are set forth in U.S. Pat. Nos. 5,817,807; 6,506,770; 6,756,391; 7,160,872; 6,872,714; 7,414,065; 6,667,320 and 7,022,717, incorporated herein by reference. In certain other embodiments, Z is 1,4,8,11-tetraazacyclotetradecane, the linker is 1,3- or 1,4-phenylene-bis(alkylene) in particular 1,4-phenylene-bis(methylene) and Z′ is —NR(CR₂)ₙ—X, where X is pyridine, and in particular wherein Z′ is NHCH₂CH₂-pyridine. In further embodiments, the compound is N-[1,4,8,11-tetraazacyclotetradecanyl-(1,4-phenylene-bis-(methylene)]-2-aminoalkylpyridine, sometimes designated herein as AMD3465.

In certain embodiments, the CXCR4 antagonist includes, but is not limited to, linear peptides, cyclic peptides, natural amino acids, unnatural amino acids, and peptidomimetic compounds. Examples of such compounds include T22 (Murakami, T., et al., J. Exp. Med. (1997) 186:1389-1393); T134 (Arakaki, R., et al., J. Virol. (1999) 73:1719-1723; T140 (Tamamura, H., et al., Biochem. Biophys. Res. Comm. (1998) 253:877-882) and its analogs TC14012 and TN14003 (Tamamura, H., et al., Bioorg. Med. Chem. Lett. (2001) 11:1897-1902; Mori, T., et al., Mol. Cancer Ther. (2004) 3:29-37; Burger, M., et al., Blood (2005) 106:1824-1830); ALX40-4C (Doranz, B. J., et al., J. Exp. Med. (1997) 186:1395-1400; Donzella, G. A., Nat. Med. (1998) 4:72-77; Doranz, B. J., et al., AIDS Res. Hum. Retrovir. (2001) 17:475-486); RCP168 (Zeng, Z., et al., Mol. Cancer Ther. (2006) 5:3113-3121); CTCE-0021 (Pelus, L. M., et al., Exp. Hematol. (2005) 33:295-307); CTCE-0214 and CTCE-9908 (Zhong, R., et al., Exp. Hematol. (2004) 32:470-475; Kim, S. Y., et al., AACR Meeting Abstracts (2005) Abstract 256; PCT Pub. Nos. WO 01/76615 and WO 01/85196; U.S. Patent Pub. No. 2007/0160574 and related applications); KRH-1120 (Yamamoto, N., et al., J. AIDS Res. (2000) 2:453-460); KRH-1636 (Ichiyama, K., et al., Proc. Natl. Acad. Sci. USA (2003) 100:4185-4190); KRH-2731/CS-3955 (Murakami T., et al., Abstracts of the 11th Conference on Retroviruses and Opportunistic Infections (2004) Abstract 541; PCT Pub. Nos. WO 06/095542 and WO 02/094261); and CXCR4 antagonists described in PCT Pub. Nos. WO 99/47158; WO 99/50461; WO 00/09152; WO 01/94420; and WO 03/090512.

In certain other embodiments, the CXCR4 antagonist is BKT140, including those CXCR4 antagonists described in U.S. Pat. No. 7,423,007 and U.S. Patent Application Pub. No. 2004/0171552; AVR 118; TG-0054, including those CXCR4 antagonists described in U.S. Pat. No. 7,399,776 and U.S. Patent Pub. Nos. 2006/0160860 and 2008/0058382; MSX-122; or POL-6326/POL-2438/POL-3026, including those CXCR4 antagonists described in PCT Pub. No. WO 2008/104090. In certain embodiments, the antagonist may be an antibody, such as a monoclonal antibody, or immunoreactive fragment thereof. The contents of all the foregoing documents are hereby incorporated herein by reference for all purposes.

Methods to synthesize certain of the CXCR4 antagonists disclosed herein are set forth in the U.S. patents and applications above as well as U.S. Pat. No. 6,489,472, PCT Pub. No. WO 02/026721 and certain other documents mentioned herein, which are incorporated herein by reference. Additional suitable CXCR4 antagonists are set forth in Appendix A.

The compounds of the invention may be prepared in the form of prodrugs, i.e., protected forms which release the compounds of the invention after administration to the subject. Typically, the protecting groups are hydrolyzed in body fluids such as in the bloodstream thus releasing the active compound or are oxidized or reduced in vivo to release the active compound. A discussion of prodrugs is found in Smith and Williams Introduction to the Principles of Drug Design, Smith, H. J.; Wright, 2^{nd} ed., London (1988).

Compounds useful in the invention, which are amines, may be administered or prepared in the forms of their acid addition salts or metal complexes thereof. Suitable acid addition salts include salts of inorganic acids that are biocompatible, including HCl, HBr, sulfuric, phosphoric and the like, as well as organic acids such as acetic, propionic, butyric and the like, as well as acids containing more than one carboxyl group, such as oxalic, glutaric, adipic and the like. Typically, at physiological pH, the compounds of the invention will be in the forms of the acid addition salts.

Compounds useful in the invention that are carboxylic acids or otherwise acidic may be administered or prepared in forms of salts formed from inorganic or organic bases that are physiologically compatible. Thus, these compounds may be prepared in the forms of their sodium, potassium, calcium, or magnesium salts as appropriate or may be salts with organic bases such as caffeine or ethylamine. These compounds also may be in the form of metal complexes.

When prepared as purified forms, the compounds may also be crystallized as the hydrates or other solvates. Those forms of the compounds used in the invention that contain chiral centers may be optically pure or may contain a mixture of stereoisomers, including racemic mixtures or mixtures of varying optical purity.

The CXCR4 antagonists may be formulated for administration to animal subject using commonly understood formulation techniques well known in the art. Formulations which are suitable for particular modes of administration and for compounds useful in the invention may be found in Remington's The Science and Practice of Pharmacy, 21^{st} edition, Lippincott Williams & Wilkins, Hagerstown, Md.

The CXCR4 antagonists may be administered by injection, such as by intravenous injection, subcutaneous or intraperitoneal injection, and the like. Additional parenteral routes of administration include intramuscular and intraarticular injection. For intravenous or parenteral administration, the compounds are formulated in suitable liquid form with excipients as required. The compositions may contain liposomes or other suitable carriers. For injection intravenously, the solution is made isotonic using standard preparations such as Hank's solution.

Besides injection, other routes of administration may also be used. The compounds may be formulated into tablets, capsules, syrups, powders, or other suitable forms for administration orally. By using suitable excipients, these compounds may also be administered through the mucosa using suppositories or intranasal sprays. Transdermal administration can also be effected by using suitable penetrants and controlling the rate of release.

The formulation and route of administration chosen will be tailored to the individual subject, the nature of the condition to be treated in the subject, and generally, the judgment of the attending practitioner.

The CXCR4 antagonists may be administered as a single bolus dose, a dose over time, as in intravenous or transdermal administration, or in multiple dosages. Suitable dosage ranges for the CXCR4 antagonists vary according to these considerations, but in general, the compounds are administered in the range of about 0.1 μg/kg-10 mg/kg of body weight; preferably the range is about 1 μg/kg-500 μg/kg up to 1 mg/kg of body weight. For a typical 70-kg human subject, thus, the dosage range is from about 7 μg to about 700 mg, preferably from about 70 μg to about 70 mg. In certain embodiments, the CXCR4 antagonist is administered or prepared in a pharmaceutical preparation at a “low dose” of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg and 1 mg/kg. In certain other embodiments, the dose of the CXCR4 antagonist is greater than 1 mg/kg, such as 2 mg/kg, 3 mg/kg, 4 mg/kg, 5 mg/kg, 6 mg/kg, 7 mg/kg, 8 mg/kg, 9 mg/kg and 10 mg/kg. Dosages may be higher when the compounds are administered orally or transdermally as compared to, for example, intravenous administration.

Use of a wide variety of immunotherapeutic agents in combination with the CXCR4 antagonists is contemplated by the present invention. Such immunotherapeutic agents include, but are not limited to, CAMPATH® (alemtuzumab), RITUXAN® (rituximab), MYLOTARG® (gemtuzumab ozogamicin), ZEVALIN® (ibritumomab tiuxetan) and BEXXAR® (tositumomab). In certain embodiments, the immunotherapeutic agent is an unconjugated, or “naked”. Examples of FDA-approved naked antibodies include CAMPATH® (alemtuzumab) and RITUXAN® (rituximab).

CAMPATH® (alemtuzumab, Genzyme, Cambridge, Mass.) is a humanized IgG1 kappa monoclonal antibody directed against the 21-28 kDa cell surface glycoprotein CD52, which is expressed on the surface of normal and malignant B and T lymphocytes, natural killer (NK) cells, monocytes, macrophages, and tissues of the male reproductive system. Alemtuzumab is indicated as a single agent for the treatment of B-cell chronic lymphocytic leukemia (B-CLL). Compositions and methods of using alemtuzumab are disclosed in detail in U.S. Pat. Nos. 5,545,403; 5,545,405; 5,654,403; and 5,846,534, and 6,569,430, all of which are incorporated herein by reference in their entireties.

RITUXAN® (rituximab, Biogen Idec, Cambridge, Mass. and Genentech, South San Francisco, Calif.) is a chimeric murine/human IgG1 kappa monoclonal antibody directed against the CD20 antigen found on the surface of normal and malignant B lymphocytes. Rituximab is indicated as a single agent for relapsed or refractory, low-grade or follicular, CD20-positive, B-cell non-Hodgkin's lymphoma (NHL) and for non-progressing, low-grade, CD20-positive B-cell NHL after first-line chemotherapy. Rituximab is also indicated in combination with various chemotherapy regimens for previously untreated follicular, CD20-positive, B-cell NHL and for previously untreated diffuse large B-cell, CD20-positive NHL.

In contrast to naked antibodies, conjugated antibodies (also known as “tagged,” “labeled,” or “loaded” antibodies) are joined to various drugs, toxins or radioactive substances. The antibodies in this context are used as delivery vehicles to take these substances directly to the cancer cells, which lessens the damage to healthy cells in other parts of the body. Depending on the nature of the “label,” conjugated antibodies are referred to as radiolabeled (a radioactive isotope), chemolabeled (a chemotherapy drug), or immunotoxins (a bacterial or plant toxin). Examples of FDA-approved radiolabeled antibodies include ZEVALIN® (ibritumomab tiuxetan) and BEXXAR® (tositumomab). At this time, the only FDA-approved immunotoxin is MYLOTARG® (gemtuzumab ozogamicin).

ZEVALIN® (ibritumomab tiuxetan, Cell Therapeutics, Seattle, Wash.) is a radiolabeled murine IgG1 kappa monoclonal antibody directed against CD20. The antibody ibritumomab is used in conjunction with the chelator tiuxetan, to which a radioactive isotope (either Yttrium-90 or Indium-111) is chelated. Ibritumomab tiuxetan is indicated for relapsed or refractory, low-grade or follicular B-cell non-Hodgkin's lymphoma (NHL), including patients with rituximab refractory follicular NHL. The ibritumomab tiuxetan therapeutic regimen has also been approved for the treatment of relapsed or refractory, rituximab-naïve, low-grade and follicular NHL.

BEXXAR® (tositumomab, GlaxoSmithKline, Research Triangle Park, N.C.) is a murine IgG2a lambda monoclonal antibody directed against the CD20 antigen. Iodine I-131 tositumomab is tositumomab labeled with a radioactive iodine isotope (I¹³¹). The tositumomab/iodine I-131 tositumomab therapeutic regimen is indicated for relapsed or refractory, CD20-positive, low-grade, follicular non-Hodgkin's lymphoma (NHL) or follicular NHL that has transformed to a faster-growing form. Tositumomab/iodine I-131 tositumomab has been approved for patients who have received chemotherapy, rituximab, or a combination thereof.

MYLOTARG® (gemtuzumab ozogamicin, Wyeth, Madison, N.J.) is a humanized IgG4 kappa monoclonal antibody conjugated with a cytotoxic antitumor antibiotic, calicheamicin, isolated from fermentation of a bacterium, Micromonospora echinospora subsp. calichensis. The antibody portion of gemtuzumab ozogamicin binds specifically to the CD33 antigen, a sialic acid-dependent adhesion protein found on the surface of leukemic blasts and immature normal cells of myelomonocytic lineage, but not on normal hematopoietic stem cells. Gemtuzumab ozogamicin is indicated for the treatment of patients with CD33⁺ acute myelogenous leukemia (AML) in first relapse who are 60 years of age or older and who are not considered candidates for other cytotoxic chemotherapy.

As noted above, AMD3100 and AMD3465 are exemplary antagonists of the CXCR4 chemokine receptor (Gerlach, et al., J. Biol. Chem. (2001) 276:14153-14160; Hatse, S., et al., Biochem. Pharmacol. (2005) 70:752-61). Accordingly, in certain embodiments, AMD3100 and AMD3465 may be used in conjunction with one or more immunotherapeutic agent(s) such as, for example, CAMPATH® (alemtuzumab), RITUXAN® (rituximab), MYLOTARG® (gemtuzumab ozogamicin), ZEVALIN® (ibritumomab tiuxetan), or BEXXAR® (tositumomab), to treat a subject afflicted with a hematological malignancy.

A wide variety of immunotherapeutic protocols may be employed, many of such protocols involving combinations of drugs administered simultaneously or in tandem. The CXCR4 antagonists may be administered at various points in the simultaneous or tandem protocols. In certain embodiments, the CXCR4 antagonist may be administered several hours before the first administration of the immunotherapeutic agent, which is repeated over several days. In certain other embodiments, the CXCR4 antagonist may be administered daily before, during, or after the administration of the immunotherapeutic agent. Various combinations of the foregoing agents may be used in such protocols, and the timing and frequency of CXCR4 administration is subject to routine optimization, within ordinary skill. Dosage levels and mode of administration are interdependent. When given subcutaneously, for example, the dosage levels are in the range of 50 μg/kg-1 mg/kg, preferably 200 μg/kg-500 μg/kg.

In certain embodiments, the present methods may further comprise administration of other mobilizing agents, immunomodulatory agents, or other nutritional or therapeutically beneficial agents. The additional factor(s) may be administered in the same composition, in different compositions but simultaneously, or in a tandem protocol with the administration of the CXCR4 antagonist. Among additional factors that can be included are recombinant G-CSF such as NEUPOGEN® (filgrastim), GRANOCYTE®/NEUTROGIN® (lenograstim) and STEMGEN® (ancestim), a covalent conjugate of recombinant G-CSF such as NEULASTA® (pegfilgrastim), granulocyte-macrophage colony stimulating factor (GM-CSF) such as LEUKINE® (sargramostim) and LEUCOMAX® (molgramostim), interleukin-1 (IL-1), interleukin-3 (IL-3), interleukin-8 (IL-8), PIXY-321 (GM-CSF/IL-3 fusion protein), REVIMID™ (CC-5013), ACTIMID™ (CC-4047), macrophage inflammatory protein, stem cell factor and thrombopoietin. In certain embodiments, the presently disclosed methods further comprise the administration of one or more of antibiotics, vitamins, herbal extracts, anti-inflammatories, nutrients, antipyretics, analgesics, cyclophosphamide and the like.

Subjects that will respond favorably to the method of the invention include medical and veterinary subjects generally, including human patients. Among other subjects for whom the methods of the invention is useful are cats, dogs, large animals, avians such as chickens, and the like, other than standard research rodents such as laboratory mice, rabbits, or rats. In general, any subject that exhibits a hematopoietic or myeloid malignancy would benefit from the methods of the invention. In certain embodiments, the subject of the treatment may further undergo bone marrow transplantation.

In an additional aspect, the present invention is directed to a pharmaceutical or veterinary composition comprising a CXCR4 antagonist in unit dosage form for use in the methods of the invention. The composition comprises a CXCR4 antagonist along with an immunotherapeutic agent, such as, for example, a therapeutic antibody, and a suitable pharmaceutically or veterinary acceptable excipient.

Formulations that are suitable for particular modes of administration and for compounds useful in the invention may be found in Remington's The Science and Practice of Pharmacy, 21^{st} edition, Lippincott Williams & Wilkins, Hagerstown, Md.

In certain embodiments, the pharmaceutical or veterinary composition may comprise a CXCR4 antagonist of formula (1) as set forth above. In some specific embodiments, the pharmaceutical or veterinary composition may comprise

1,1′-[1,4-phenylene-bis-(methylene)-bis-1,4,8,11-tetraazacyclotetradecane (AMD3100) and N-[1,4,8,11-tetraazacyclotetradecanyl-(1,4-phenylene-bis-(methylene)]-2-aminoethyl-2-pyridine (AMD3465).

In certain embodiments, the immunotherapeutic agent may comprise a human monoclonal antibody, a mouse monoclonal antibody, a chimeric monoclonal antibody, a humanized monoclonal antibody or a combination thereof.

In certain embodiments, the immunotherapeutic agent may comprise an unconjugated antibody, a radiolabeled antibody, a chemolabeled antibody, an immunotoxin (i.e., a toxin-labeled antibody) or a combination thereof.

In certain embodiments, the immunotherapeutic agent may comprise CAMPATH® (alemtuzumab), RITUXAN® (rituximab), MYLOTARG® (gemtuzumab ozogamicin), ZEVALIN® (ibritumomab tiuxetan), BEXXAR® (tositumomab) or a combination thereof.

In some specific embodiments, AMD3100 or AMD3465 may be used in conjunction with CAMPATH® (alemtuzumab), RITUXAN® (rituximab), MYLOTARG® (gemtuzumab ozogamicin), ZEVALIN® (ibritumomab tiuxetan), BEXXAR® (tositumomab) or a combination thereof.

Having now generally described the invention, the same will be more readily understood through reference to the following examples which are provided by way of illustration, and are not intended to be limiting of the present invention, unless specified.

### Example 1

### Efficacy of AMD3465 in the Disseminated Raji Lymphoma Model

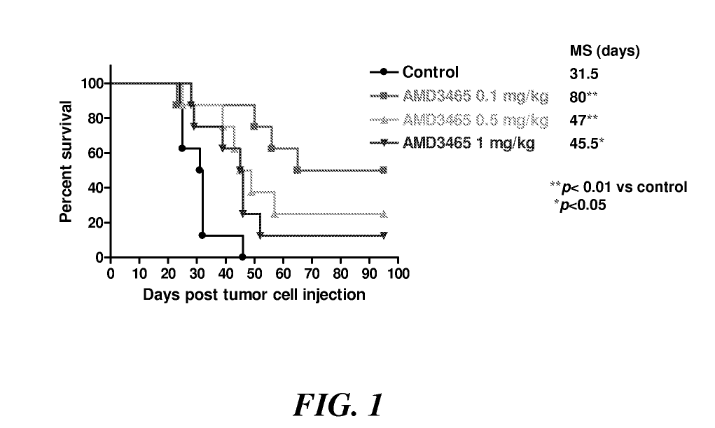

The in vivo therapeutic efficacy of the CXCR4 antagonist AMD3465 was studied in a severe combined immunodeficient (SCID) mouse lymphoma model. Four groups of 4- to 6-week-old SCID mice (8 animals each) were injected intravenously with 2×10⁶ Raji B-cell lymphoma cells. The Raji cell line is a well-characterized human B-cell lymphoblastic line (CXCR4⁺, CD19⁺, CD20⁺, CD22⁺, CD52⁺) derived from a patient with Burkitt's lymphoma (available from the American Tissue and Cell Collection, Manassas, Va.). Starting on day 7 after the injection, three of the four groups were administered subcutaneous AMD3465 daily (Monday-Friday regimen) at 0.1 mg/kg, 0.5 mg/kg or 1.0 mg/kg body weight. The control group did not receive any AMD3465. The experimental setup is summarized in Table 1. The mean survival in each group was estimated by the Kaplan-Meier method, as shown in FIG. 1.

TABLE 1

| Group | | Animals |
|---|---|---|
| # | Treatment Group | per group |
| 1 | 2 × 10⁶ Raji cells injected, Control | 8 |
| 2 | 2 × 10⁶ Raji cells + AMD3465 at 0.1 mg/kg | 8 |
| | beginning on day 7 post-injection (M-F regimen) | |
| 3 | 2 × 10⁶ Raji cells + AMD3465 at 0.5 mg/kg | 8 |
| | beginning on day 7 post-injection (M-F regimen) | |
| 4 | 2 × 10⁶ Raji cells + AMD3465 at 1.0 mg/kg | 8 |
| | beginning on day 7 post-injection (M-F regimen) | |

As shown in FIG. 1, AMD3465 alone significantly increased the mean survival in the disseminated Raji lymphoma model at each concentration tested, with a surprisingly stronger effect observed at the lower concentrations (i.e., reverse dose-response).

### Example 2

### Efficacy of CAMPATH® (Alemtuzumab) in Combination with AMD3465 in the Disseminated Raji Lymphoma Model

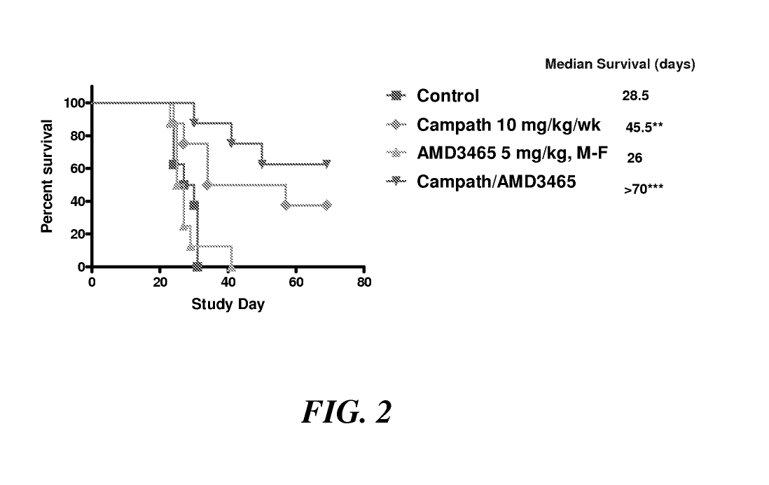

The in vivo therapeutic efficacy of CAMPATH® (alemtuzumab) in combination with the CXCR4 antagonist AMD3465 was studied in a severe combined immunodeficient (SCID) mouse lymphoma model substantially as described above. Four groups of 4- to 6-week-old SCID mice (8 animals each) were injected intravenously with 2×10⁶ Raji B-cell lymphoma cells. Starting on day 7 after the injection, one group was administered CAMPATH® (alemtuzumab) weekly at 10 mg/kg; a second group was administered AMD3465 daily (Monday-Friday regimen) at 0.5 mg/kg; and a third group was administered CAMPATH® (alemtuzumab) weekly at 10 mg/kg and AMD3465 daily (Monday-Friday regimen) at 0.5 mg/kg body weight. The control group did not receive any AMD3465 or CAMPATH® (alemtuzumab). The experimental setup is summarized in Table 2. The mean survival in each group was estimated by the Kaplan-Meier method, as shown in FIG. 2.

TABLE 2

| Group | | Animals |
|---|---|---|
| # | Treatment Group | per group |
| 1 | 2 × 10⁶ Raji cells injected, Control | 8 |
| 2 | 2 × 10⁶ Raji cells + CAMPATH ® (alemtuzumab) | 8 |
| | at 10 mg/kg weekly beginning on day 7 post- | |
| | injection | |
| 3 | 2 × 10⁶ Raji cells + AMD3465 at 5.0 mg/kg daily | 8 |
| | (M-F regimen) beginning on day 7 post-injection | |
| 4 | 2 × 10⁶ Raji cells + CAMPATH ® (alemtuzumab) | 8 |
| | at 10 mg/kg weekly and AMD4365 at 5 mg/kg daily | |
| | (M-F regimen) beginning on day 7 post- | |
| | injection | |

As shown in FIG. 2, the combination of 5.0 mg/kg AMD3465 and CAMPATH® (alemtuzumab) significantly increased the mean survival in the disseminated Raji tumor model compared to CAMPATH® (alemtuzumab) alone. Consistent with the reverse AMD3465 dose response observed in Example 1, treatment with 5.0 mg/kg AMD3465 alone did not have a significant effect on the mean survival.

### Example 3

### Efficacy of RITUXAN® (Rituximab) in Combination with AMD3100 in the Disseminated Raji Lymphoma Model

The in vivo therapeutic efficacy of RITUXAN® (rituximab) in combination with the CXCR4 antagonist AMD3100 was studied in a severe combined immunodeficient (SCID) mouse lymphoma model substantially as described above. Four groups of 4- to 6-week-old SCID mice (8 animals each) were injected intravenously with 2×10⁶ Raji B-cell lymphoma cells. Starting on day 7 after the injection, one group was administered RITUXAN® (rituximab) twice a week (Monday and Friday regimen) at 10 mg/kg; a second group was administered AMD3100 three times a week (Monday, Wednesday, Friday regimen) at 1.0 mg/kg; and a third group was administered RITUXAN® (rituximab) twice a week (Monday and Friday regimen) at 10 mg/kg and AMD3100 three times a week (Monday, Wednesday, Friday regimen) at 1.0 mg/kg body weight. The control group did not receive any AMD3100 or RITUXAN® (rituximab). The experimental setup is summarized in Table 3.

TABLE 3

| Group | | Animals |
|---|---|---|
| # | Treatment Group | per group |
| 1 | 2 × 10⁶ Raji cells injected, Control | 8 |
| 2 | 2 × 10⁶ Raji cells + RITUXAN ® (rituximab) | 8 |
| | at 10 mg/kg twice a week (M, F regimen) | |
| | beginning on day 7 post-injection | |
| 3 | 2 × 10⁶ Raji cells + AMD3100 at 1.0 mg/kg | 8 |
| | thee times a week (M, W, F regimen) beginning on | |
| | day 7 post-injection | |
| 4 | 2 × 10⁶ Raji cells + RITUXAN ® (rituximab) | 8 |
| | at 10 mg/kg (M, F regimen) + AMD3100 at | |
| | 1.0 mg/kg (M, W, F regimen) beginning on day 7 | |
| | post-injection | |

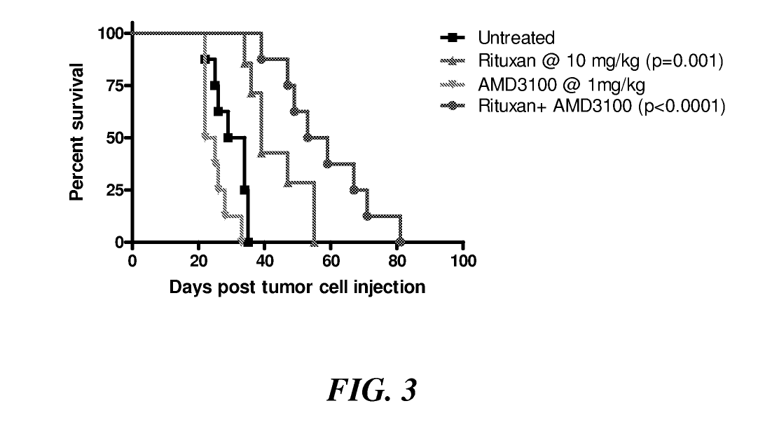

As shown in FIG. 3, the combination of 1.0 mg/kg AMD3100 and RITUXAN® (rituximab) significantly increased survival in the disseminated Raji lymphoma model compared to RITUXAN® (rituximab) alone.

### Example 4

### Efficacy of CAMPATH® (Alemtuzumab) in Combination with AMD3465 in the Disseminated B104 Lymphoma Model

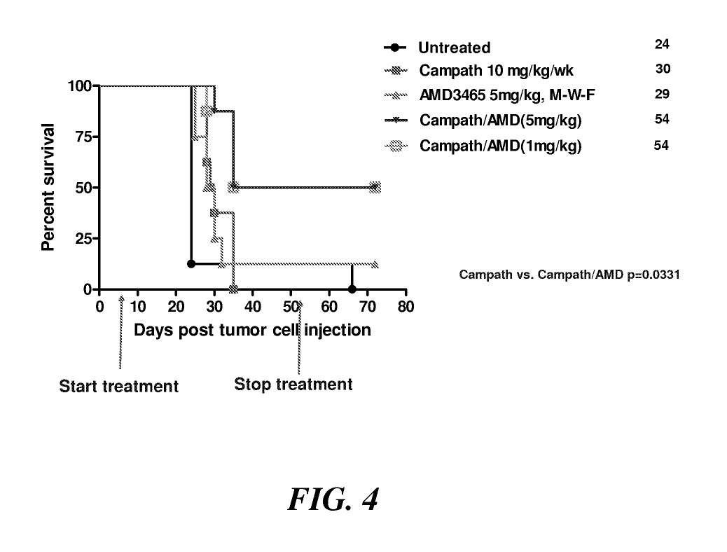

The in vivo therapeutic efficacy of the CXCR4 antagonist AMD3465 was studied in an alternative severe combined immunodeficient (SCID) mouse lymphoma model. Five groups of 4- to 6-week-old SCID mice (8 animals each) were injected intravenously with 1×10⁶ B104 B-cell lymphoma cells. The B104 cell line is a human B-cell lymphoma line that also expresses high levels of CXCR4, CD20 and CD52 (available from the Japanese Collection of Research Bioresources, Osaka, Japan). Starting on day 7 after the injection, one group was administered CAMPATH® (alemtuzumab) weekly at 10 mg/kg; a second group was administered AMD3465 three times a week (Monday, Wednesday, Friday regimen) at 5.0 mg/kg; a third group was administered CAMPATH® (alemtuzumab) weekly at 10 mg/kg and AMD3465 three times a week (Monday, Wednesday, Friday regimen) at 1.0 mg/kg; and a fourth group was administered CAMPATH® (alemtuzumab) weekly at 10 mg/kg and AMD3465 three times a week (Monday, Wednesday, Friday regimen) at 5.0 mg/kg. The control group did not receive any AMD3465 or CAMPATH® (alemtuzumab). The treatment was terminated on day 42. The experimental setup is summarized in Table 4. The mean survival in each group was estimated by the Kaplan-Meier method, as shown in FIG. 4.

TABLE 4

| Group | | Animals |
|---|---|---|
| # | Treatment Group | per group |
| 1 | 1 × 10⁶ B104 cells injected, Control | 8 |
| 2 | 1 × 10⁶ B104 cells + CAMPATH ® (alemtuzumab) | 8 |
| | at 10 mg/kg week beginning on day 7 post-injection | |
| 3 | 1 × 10⁶ B104 cells + AMD3465 at 5.0 mg/kg three | 8 |
| | times a week (M, W, F regimen) beginning on day 7 | |
| | post-injection | |
| 4 | 1 × 10⁶ B104 cells + CAMPATH ® (alemtuzumab) | 8 |
| | at 10 mg/kg weekly and AMD3465 at 1.0 mg/kg | |
| | three times a week (M, W, F regimen) beginning on | |
| | day 7 post-injection | |
| 5 | 1 × 10⁶ B104 cells + CAMPATH ® (alemtuzumab) | 8 |
| | at 10 mg/kg weekly and AMD3465 at 5.0 mg/kg | |
| | three times a week (M, W, F regimen) beginning on | |
| | day 7 post-injection | |

As shown in FIG. 4, the combination of AMD3465 and CAMPATH® (alemtuzumab) significantly increased survival in the disseminated B104 tumor model compared to CAMPATH® (alemtuzumab) alone at both concentrations of AMD3465 tested. Consistent with the results in Example 2, treatment with 5.0 mg/kg AMD3465 alone did not have a significant effect on the mean survival.

### Example 5

### Efficacy of CAMPATH® (Alemtuzumab) in Combination with AMD3465 in the Disseminated B104 Lymphoma Model—Alternative Regimen

This example describes another demonstration of the in vivo therapeutic efficacy of AMD3465 in combination with CAMPATH® (alemtuzumab) in severe combined immunodeficiency (SCID) mice bearing disseminated B104 lymphoma cells. Four groups of 4- to 6-week-old SCID mice (8 animals each) were injected intravenously with 1×10⁶ B104 B-cell lymphoma cells. Starting on day 10 after the injection, one group was administered CAMPATH® (alemtuzumab) weekly at 10 mg/kg; a second group was administered AMD3465 daily (Monday-Friday regimen) at 5.0 mg/kg; and a third group was administered CAMPATH® (alemtuzumab) weekly at 10 mg/kg and AMD3465 daily (Monday-Friday regimen) at 5.0 mg/kg body weight. The control group did not receive any AMD3465 or CAMPATH® (alemtuzumab). The experimental setup is summarized in Table 5.

TABLE 5

| Group | | Animals |
|---|---|---|
| # | Treatment Group | per group |
| 1 | 1 × 10⁶ B104 cells injected, Control | 8 |
| 2 | 1 × 10⁶ B104 cells + CAMPATH ® (alemtuzumab) | 8 |
| | at 10 mg/kg weekly beginning on day 10 post- | |
| | injection | |
| 3 | 1 × 10⁶ B104 cells + AMD3465 at 5.0 mg/kg | 8 |
| | daily (M-F regimen) beginning on day 10 post- | |
| | injection | |
| 4 | 1 × 10⁶ B104 cells + CAMPATH ® (alemtuzumab) | 8 |
| | at 10 mg/kg weekly and AMD4365 at 5.0 mg/kg | |
| | daily (M-F regimen) beginning on day 10 post- | |
| | injection | |

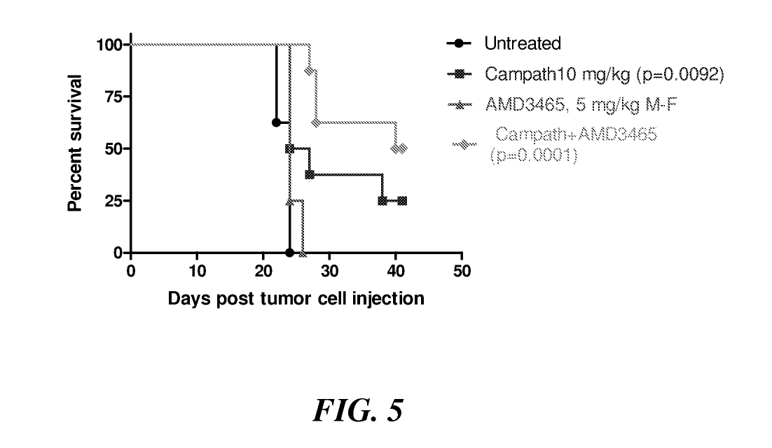

As shown in FIG. 5, the combination of 5.0 mg/kg AMD3465 and CAMPATH® (alemtuzumab) significantly increased survival in the disseminated B104 tumor model compared to CAMPATH® (alemtuzumab) alone. Consistent with the results in Examples 2 and 4, treatment with 5.0 mg/kg AMD3465 alone did not have a significant effect on the mean survival.

The results described in Examples 1-5 strongly suggest that there is a potential role for CXCR4 antagonists in combination with immunotherapeutic agents in the treatment of hematological malignancies.

### APPENDIX A

Exemplary CXCR4 antagonists of Formula 1 include compounds of formula (1A):

V—CR₂—Ar¹—CR₂NR—(CR₂)ₓ—Ar² (1A)
wherein V is a substituted heterocycle of 9-24 members containing 2-4 optionally substituted amine nitrogen atoms spaced from each other by 2 or more optionally substituted carbon atoms, and which heterocycle may optionally comprise a fused aromatic or heteroaromatic ring, and wherein
(a) said heterocycle contains at least one O or S, said O or S spaced from any adjacent heteroatom by at least 2 carbon atoms, and wherein said S is optionally oxidized or
(b) at least one carbon atom in said ring is substituted by an electron-withdrawing substituent, or
(c) both (a) and (b);
and wherein each R is independently H or a straight chain, branched or cyclic alkyl containing 1-6C;
x is 0-4;
Ar¹ is an unsubstituted or substituted aromatic or heteroaromatic moiety; and
Ar² is an unsubstituted or substituted aromatic or heterocyclic group.

In another embodiment of Formula 1, the CXCR4 antagonist has formula

V—CH₂—Ar¹—CH₂NR—CH₂—Ar²
wherein V is a heterocycle as defined in formula (1A), and wherein:
(a) said heterocycle is substituted with halo or ═O; or
(b) said heterocycle contains O or S; or
(c) both (a) and (b),
and wherein Ar¹ is unsubstituted 1, 3 or 1,4-phenylene, R is H, methyl or ethyl and Ar² is unsubstituted phenyl or pyridinyl. Preferred embodiments of x are 0-2 and 1-2.

The heterocycle V may contain 3 N and at least one carbon atom in the heterocycle that is substituted by at least one fluoro substituent. The R moiety may independently be hydrogen or methyl. The number of (CR₂)ₓ groups may be 0-4, 0-2, or 1-2. The Ar¹ moiety may be 1, 3 or 1,4-phenylene. The Ar² moiety may be phenyl or pyridyl. The heterocycle V may be a 12-16 membered heterocycle, or may contain O or S as a ring member. The heterocycle V may also contain an oxidized sulfur as a ring member. In one example, at least one carbon in the heterocycle V is substituted by ═O.

Compounds of formula (1A) and methods of synthesizing such compounds are described in PCT Pub. No. WO 01/44229 and U.S. Pat. Nos. 6,667,320 and 7,022,717, incorporated herein by reference.

Related to these compounds having formula (1B):

V—CR¹R²—Ar—CR³R⁴—N(R⁵)—(CR⁶R⁷)ₓ—R⁸ (1B)
wherein V is an optionally substituted 1,4,8,11-tetraazacyclotetra-decanyl, 4,7,10,17-tetraazabicyclo[13.3.1]heptadeca-1(17),13,15-trienyl, 1,4,7-triazacyclotetra-decanyl, 4,7,10-triazabicyclo[13.3.1]heptadeca-1(17),13,15-trienyl, 1,7-diazacyclotetradecanyl, or 4,10-diazabicyclo[13.31.1]heptadeca-1(17),13,15-trienyl system;
R¹ to R⁷ may be the same or different and are independently selected from hydrogen or straight, branched or cyclic C1-6 alkyl;
R⁸ is pyridyl, pyrimidinyl, pyrazinyl, imidazolyl, thiophene-yl, thiophenyl, aminobenzyl, piperidinyl, purine, piperazinyl, phenylpiperazinyl, or mercaptan;
Ar is a phenylene ring optionally substituted at single or multiple positions with alkyl, aryl, amino, alkoxy, hydroxy, halogen, carboxyl and/or carboxamido; and
x is 1 or 2.

In the above formula (1B), the V moiety may be optionally substituted by hydroxyl, alkoxy, thiol, thioalkyl, halogen, nitro, carboxy, amido, sulfonic acid, and/or phosphate.

Compounds of formula (1B), pharmaceutically acceptable salts or metal complexes thereof, and methods of synthesizing such compounds are described in PCT Pub. No. WO 00/02870 and U.S. Pat. No. 5,817,807, incorporated herein by reference.

Other CXCR₄ antagonists are of formula (1C):

V²—CR₉R₁₀—Ar² (1C)
wherein V² is an optionally substituted 1,4,8,11-tetraazacyclotetra-decanyl or 4,7,10,17-tetraazabicyclo[13.3.1]heptadeca-1(17),13,15-trienyl system;
R₉ and R₁₀ may be the same or different and are independently selected from hydrogen or straight, branched or cyclic C₁₋₆ alkyl;
Ar² is an aromatic or heterocyclic ring each optionally substituted at single or multiple positions with electron-donating or withdrawing groups and/or aromatic and heterocyclic groups and their alkyl derivatives thereof, and the acid addition salts and metal complexes.

In the above formula (1C), Ar₂ may be optionally substituted with alkyl, aryl, amino, alkoxy, hydroxy, halogen, carboxyl and/or carboxamido. In particular examples, Ar₂ is optionally substituted with alkoxy, alkyl, or halogen.

Compounds having formula (1C), and methods of synthesizing the same, are described in PCT Pub. No. WO 00/02870 and U.S. Pat. Nos. 6,506,770; 6,756,391; 7,160,872; 6,872,714; and 7,414,065, incorporated herein by reference.

Other CXCR4 antagonists are of formula (1D):

V—R-A-R—W (1D)
wherein V and W are independently cyclic polyamine moieties having from 9 to 32 ring members and from 3 to 8 amine nitrogens in the ring spaced by 2 or more carbon atoms from each other, and having one or more aromatic or heteroaromatic rings fused thereto,
A is an aromatic or heteroaromatic moiety when V and W have one or more aromatic or heteroaromatic moieties fused thereto, with or without an additional heteroatom other than nitrogen incorporated in the ring, or A is an aromatic or heteroaromatic moiety when V and W contain a heteroatom other than nitrogen incorporated in the ring without having one or more aromatic or heteroaromatic moieties fused thereto,
and R and R′ are each a substituted or unsubstituted alkylene chain or heteroatom-containing chain which spaces the cyclic polyamines and the moiety A.

In the above Formula (1D), R and R′ may each be methylene. In one example, A is 1,3- or 1,4-phenylene. In another example, each V and W is an unsubstituted or substituted tricyclic or bicyclic ring system containing only carbon and nitrogen atoms in the rings. One of the cyclic ring systems may be a 10 to 20 membered polyamine ring system having from 3 to 6 amine nitrogen atoms, and the ring system or systems is a fused benzyl or pyridinyl ring system.

Compounds having formula (1D), and methods of synthesizing such compounds, are described in U.S. Pat. No. 5,698,546, incorporated herein by reference.

Other CXCR4 antagonists are of formula (1E):

Z—R-A-R′—Y (1E)
where Z and Y are identical cyclic polyamine moieties having from 10 to 15 ring members and from 3 to 6 amine nitrogens in the ring spaced by 2 or more carbon atoms from each other, said amine nitrogens being the only ring heteroatoms,
A is an aromatic or heteroaromatic moiety other than quinoline,
R and R′ are each methylene linked to nitrogen atoms in Z and Y, the amine nitrogen atoms being otherwise unsubstituted.

In the above formula (1E), each moiety Z and Y may have 14 ring members and 4 amine nitrogens in the ring. Compounds having formula (1E), and methods of synthesizing such compounds, are described in U.S. Pat. No. 5,583,131, incorporated herein by reference.

The CXCR4 antagonist may be of formula (1F):

Z-(A)ₙ-Y (1F)
where Z and Y are independently cyclic polyamine moieties having from 9 to 32 ring members and from 3 to 8 amine nitrogen atoms in the ring,
A is a linking atom or group, and n is O or an integer from 1 to 6.

In the above formula (1F) each Z and Y moiety may have 10 to 24 ring members, or 12 to 18 ring members. Each Z and Y moiety may also have 4 to 6 amine nitrogen atoms in the ring. In one example, n is 0. In another example, A is methylene.

Compounds having formula (1F), and methods of synthesizing such compounds, are described in U.S. Pat. Nos. 5,021,409 and 6,001,826, incorporated herein by reference.

In specific embodiments, the compound of formula (1) is selected from:
3,3′-bis-1,5,9,13-tetraazacyclohexadecane;
3,3′-bis-1,5,8,11,14-pentaazacyclohexadecane;
5,5′-bis-1,4,8,11-tetraazacyclotetradecane;
2,5′-bis-1,4,8,11-tetraazacyclotetradecane;
2,6′-bis-1,4,8,11-tetraazacyclotetradecane;
methylene (or polymethylene) di 1-N-1,4,8,11-tetraazacyclotetradecane;
11,11′-(1,2-ethanediyl)bis-1,4,8,11-tetraazacyclotetradecane;
11,11′-(1,2-propanediyl)bis-1,4,8,11-tetraazacyclotetradecane;
11,11′-(1,2-butanediyl)bis-1,4,8,11-tetraazacyclotetradecane;
11,11′-(1,2-pentanediyl)bis-1,4,8,11-tetraazacyclotetradecane;
11,11′-(1,2-hexanediyl)bis-1,4,8,11-tetraazacyclotetradecane;
1,1′-[1,3-phenylene-bis(methylene)]-bis-1,4,8,11-tetraazacyclotetradecane;
1,1′-[1,4-phenylene-bis(methylene)]-bis-1,4,8,11-tetraazacyclotetradecane;
1,1′-[3,3′-biphenylene-bis-(methylene)]-bis-1,4,8,11-tetraazacyclotetradecane;
11,11′-[1,4-phenylene-bis-(methylene)]-bis-1,4,7,11-tetraazacyclotetradecane;
1,11′-[1,4-phenylene-bis(methylene)]-1,4,8,11-tetraazacyclotetradecane;
1,1′-[2,6-pyridine-bis-(methylene)]-bis-1,4,8,11-tetraazacyclotetradecane;
1,1-[3,5-pyridine-bis-(methylene)]-bis-1,4,8,11-tetraazacyclotetradecane;
1,1′-[2,5-thiophene-bis-(methylene)]-bis-1,4,8,11-tetraazacyclotetradecane;
1,1′-[4,4′-(2,2′-bipyridine)-bis-(methylene)]-bis-1,4,8,11-tetraazacyclotetradecane;
1,1′-[2,9-(1,10-phenanthroline)-bis-(methylene)]-bis-1,4,8,11-tetraazacyclotetradecane;
1,1′-[1,3-phenylene-bis-(methylene)]-bis-1,4,7,10-tetraazacyclotetradecane;
1,1′-[1,4-phenylene-bis-(methylene)]-bis-1,4,7,10-tetraazacyclotetradecane;
1,1′-[5-nitro-1,3-phenylenebis(methylene)]bis-1,4,8,11-tetraazacyclotetradecane;
1,1′-[2,4,5,6-tetrachloro-1,3-phenyleneis(methylene)]bis-1,4,8,11-tetraazacyclotetradecane;
1,1′-[2,3,5,6-tetrafluoro-1,4-phenylenebis(methylene)]bis-1,4,8,11-tetraazacyclotetradecane;
1,1′-[1,4-naphthylene-bis-(methylene)]bis-1,4,8,11-tetraazacyclotetradecane;
1,1′-[1,3-phenylenebis-(methylene)]bis-1,5,9-triazacyclododecane;
1,1′-[1,4-phenylene-bis-(methylene)]-1,5,9-triazacyclododecane;
1,1′-[2,5-dimethyl-1,4-phenylenebis-(methylene)]-bis-1,4,8,11-tetraazacyclotetradecane;
1,1′-[2,5-dichloro-1,4-phenylenebis-(methylene)]-bis-1,4,8,11-tetraazacyclotetradecane;
1,1′-[2-bromo-1,4-phenylenebis-(methylene)]-bis-1,4,8,11-tetraazacyclotetradecane;
1,1′-[6-phenyl-2,4-pyridinebis-(methylene)]-bis-1,4,8,11-tetraazacyclotetradecane;
7,7′-[1,4-phenylene-bis(methylene)]bis-3,7,11,17-tetraazabicyclo[13.3.1]heptadeca-1(17),13,15-triene;
7,7′-[1,4-phenylene-bis(methylene)]bis[15-chloro-3,7,11,17-tetraazabicyclo[13.3.1]heptadeca-1(17),13,15-triene];
7,7′-[1,4-phenylene-bis(methylene)]bis[15-methoxy-3,7,11,17-tetraazabicyclo[13.3.1]heptadeca-1(17),13,15-triene];
7,7′-[1,4-phenylene-bis(methylene)]bis-3,7,11,17-tetraazabicyclo[13.3.1]-heptadeca-13,16-triene-15-one;
7,7′-[1,4-phenylene-bis(methylene)]bis-4,7,10,17-tetraazabicyclo[13.3.1]-heptadeca-1(17),13,15-triene;
8,8′-[1,4-phenylene-bis(methylene)]bis-4,8,12,19-tetraazabicyclo[15.3.1]nonadeca-1(19),15,17-triene;
6,6′-[1,4-phenylene-bis(methylene)]bis-3,6,9,15-tetraazabicyclo[11.3.1]pentadeca-1(15),11,13-triene;
6,6′-[1,3-phenylene-bis(methylene)]bis-3,6,9,15-tetraazabicyclo[11.3.1]pentadeca-1(15),11,13-triene;
17,17′-[1,4-phenylene-bis(methylene)]bis-3,6,14,17,23,24-hexaazatricyclo[17.3.1.18,12]tetracosa-1(23),8,10,12(24),19,21-hexaene;
N-[1,4,8,11-tetraazacyclotetradecanyl-1,4-phenylenebis(methylene)]-2-(amino-methyl)pyridine;
N-[1,4,8,11-tetraazacyclotetradecanyl-1,4-phenylenebis(methylene)]-N-methyl-2-(aminomethyl)pyridine;
N-[1,4,8,11-tetraazacyclotetradecanyl-1,4-phenylenebis(methylene)]-4-(amino-methyl)pyridine;
N-[1,4,8,11-tetraazacyclotetradecanyl-1,4-phenylenebis(methylene)]-3-(amino-methyl)pyridine;
N-[1,4,8,11-tetraazacyclotetradecanyl-1,4-phenylenebis(methylene)]-(2-amino-methyl-5-methyl)pyrazine;
N-[1,4,8,11-tetraazacyclotetradecanyl-1,4-phenylenebis(methylene)]-2-(amino-ethyl)pyridine;
N-[1,4,8,11-tetraazacyclotetradecanyl-1,4-phenylenebis(methylene)]-2-(amino-methyl)thiophene;
N-[1,4,8,11-tetraazacyclotetradecanyl-1,4-phenylenebis(methylene)]-2-(amino-ethyl)mercaptan;
N-[1,4,8,11-tetraazacyclotetradecanyl-1,4-phenylenebis(methylene)]-2-amino-benzylamine;
N-[1,4,8,11-tetraazacyclotetradecanyl-1,4-phenylenebis(methylene)]-4-amino-benzylamine;
N-[1,4,8,11-tetraazacyclotetradecanyl-1,4-phenylenebis(methylene)]-4-(amino-ethyl)imidazole;
N-[1,4,8,11-tetraazacyclotetradecanyl-1,4-phenylenebis(methylene)]-benzylamine;
N-[1,4,8,11-tetraazacyclotetradecanyl-1,4-phenylenebis(methylene)]-purine;
N-[1,4,8,11-tetraazacyclotetradecanyl-1,4-phenylenebis(methylene)]-4-phenylpiperazine;
1-[2,6-dimethoxypyrid-4-yl(methylene)]-1,4,8,11-tetraazacyclotetradecane;
1-[2-chloropyrid-4-yl(methylene)]-1,4,8,11-tetraazacyclotetradecane;
1-[2,6-dimethylpyrid-4-yl(methylene)]-1,4,8,11-tetraazacyclotetradecane;
1-[2-methylpyrid-4-yl(methylene)]-1,4,8,11-tetraazacyclotetradecane;
1-[2,6-dichloropyrid-4-yl(methylene)]-1,4,8,11-tetraazacyclotetradecane;
1-[2-chloropyrid-5-yl(methylene)]-1,4,8,11-tetraazacyclotetradecane;
7-[4-methylphenyl(methylene)]-4,7,10,17-tetraazabicyclo[13.3.1]heptadeca-1(17),13,15-triene;
N-[4-(1,4,7-triazacyclotetra-decanyl)-1,4-phenylenebis(methylene)]-2-(aminomethyl)pyridine;
N-[1-(1,4,7-triazacyclotetra-decanyl)-1,4-phenylenebis(methylene)]-2-(aminomethyl)pyridine;
N-[7-(4,7,10,17-tetraazabicyclo[13.3.1]heptadeca-1(17),13,15-trienyl)-1,4-phenylenebis(methylene)]-2-(aminomethyl)pyridine;
N-[7-(4,7,10-triazabicyclo[13.3.1]heptadeca-1(17),13,15-trienyl)-1,4-phenylenebis(methylene)]-2-(aminomethyl)pyridine;
N-[4-[4,7,10-triazabicyclo[13.3.1]heptadeca-1(17),13,15-trienyl]-1,4-phenylenebis(methylene)]-2-(aminomethyl)pyridine;
N-[4-[4,7,10,17-tetraazabicyclo[13.3.1]heptadeca-1(17),13,15-trienyl]-1,4-phenylenebis(methylene)]-2-(aminomethyl)pyridine;
N-[3-(3,6,17-triazabicyclo[13.3.1]heptadeca-1(17),13,15-trienyl)-1,4-phenylenebis(methylene)]-2-(aminomethyl)pyridine;
N-[3-(3,6,17-triazabicyclo[13.3.1]heptadeca-1(17),13,15-trienyl)-1,3-phenylenebis(methylene)]-2-(aminomethyl)pyridine;
N-[4-(4,7,17-triazabicyclo[13.3.1]heptadeca-1(17),13,15-trienyl)-1,4-phenylenebis(methylene)]-2-(aminomethyl)pyridine;
N-[7-(4,7,17-triazabicyclo[13.3.1]heptadeca-1(17),13,15-trienyl)-1,4-phenylenebis(methylene)]-2-(aminomethyl)pyridine;
N-[6-(3,6,9-triazabicyclo[11.3.1]pentadeca-1(15),11,13-trienyl)-1,3-phenylenebis(methylene)]-2-(aminomethyl)pyridine;
N-[4-(1,7-diazacyclotetradecanyl)-1,4-phenylenebis(methylene)]-2-(aminomethyl)pyridine;
N-[7-(4,10-diazabicyclo[13.3.1]heptadeca-1(17),13,15-trienyl)-1,4-phenylenebis(methylene)]-2-(aminomethyl)pyridine;
N-[7-(4,10,17-triazabicyclo[13.3.1]heptadeca-1(17),13,15-trienyl)-1,4-phenylenebis(methylene)]-2-(aminomethyl)pyridine;
N-[4-(11-fluoro-1,4,7-triazacyclotetradecanyl)-1,4-phenylenebis(methylene)]-2-(aminomethyl)pyridine;
N-[4-(11,11-difluoro-1,4,7-triazacyclotetradecanyl)-1,4-phenylenebis(methylene)]-2-(aminomethyl)pyridine;
N-[4-(1,4,7-triazacyclotetradecan-2-onyl)-1,4-phenylenebis(methylene)]-2-(aminomethyl)pyridine;
N-[12-(5-oxa-1,9-diazacyclotetradecanyl)-1,4-phenylenebis(methylene)]-2-(aminomethyl)pyridine;
N-[4-(11-oxa-1,4,7-triazacyclotetradecanyl)-1,4-phenylenebis(methylene)]-2-(aminomethyl)pyridine;
N-[4-(11-thia-1,4,7-triazacyclotetradecanyl)-1,4-phenylenebis(methylene)]-2-(aminomethyl)pyridine;
N-[4-(11-sulfoxo-1,4,7-triazacyclotetradecanyl)-1,4-phenylenebis(methylene)]-2-(aminomethyl)pyridine;
N-[4-(11-sulfono-1,4,7-triazacyclotetradecanyl)-1,4-phenylenebis(methylene)]-2-(aminomethyl)pyridine; and
N-[4-(3-carboxo-1,4,7-triazacyclotetradecanyl)-1,4-phenylenebis(methylene)]-2-(aminomethyl)pyridine;
   or a pharmaceutically acceptable salt thereof.

It is understood that the foregoing detailed description and accompanying examples are merely illustrative, and are not to be taken as limitations upon the scope of the invention. U.S. patents and publications referenced herein are incorporated by reference.

1. A method for treating a hematological malignancy in a human comprising administering to the human a therapeutically effective amount of a CXCR4 antagonist in combination with an immunotherapeutic agent, wherein the CXCR4 antagonist comprising is a compound of the formula:
Z-linker-Z′ (1)
or a pharmaceutically acceptable salt or prodrug thereof,
wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;
Z′ may be embodied in a form as defined by Z above, or alternatively may be of the formula
—N(R)—(CR₂)ₙ—X
wherein each R is independently H or straight, branched or cyclic alkyl (1-6C), n is 1 or 2, and X is an aromatic ring, including heteroaromatic rings, or is a mercaptan,
or Z′ may be of the formula
—Ar(Y)ⱼ
wherein Ar is an aromatic or heteroaromatic moiety, and each Y is independently a non-interfering substituent and j is 0-3; and
“linker” represents a bond, alkylene (1-6C) or may comprise aryl, fused aryl, oxygen atoms contained in an alkylene chain, or may contain keto groups or nitrogen or sulfur atoms.

2. The method of claim 1, wherein Z and Z′ are both cyclic polyamines.

3. The method of claim 1, wherein Z and Z′ are identical.

4. The method of claim 1, wherein Z is a cyclic polyamine that contains 10-24 members and contains 4 nitrogen atoms.

5. The method of claim 1, wherein Z and Z′ are both 1,4,8,11-tetraazocyclotetradecane.

6. The method of claim 1, wherein the linker comprises an aromatic ring bracketed by two methylene moieties.

7. The method of claim 6, wherein the linker is 1,4-phenylene-bis-methylene.

8. The method of claim 7, wherein the compound of formula (1) is 1,1′-[1,4-phenylene-bis-(methylene)-bis-1,4,8,11-tetraazacyclotetradecane or a pharmaceutically acceptable salt thereof.

9. The method of claim 1, wherein the compound of formula (1) is in the form of an acid addition salt.

10. The method of claim 9, wherein the acid addition salt is hydrochloride.

11. The method of claim 1, wherein Z′ is of the formula
—N(R)—(CR₂)ₙ₋X
wherein each R, N and X are as defined in claim 1.

12. The method of claim 11, wherein the linker comprises an aromatic ring bracketed by two methylene moieties.

13. The method of claim 12, wherein the linker is 1,4-phenylene-bis-methylene.

14. The method of claim 11, wherein each R is H, n is 2 and X is substituted or unsubstituted pyridyl.

15. The method of claim 11, wherein Z′ is 2-aminomethyl-pyridine.

16. The method of claim 15, wherein the compound of formula (1) is N-[1,4,8,11-tetraazacyclotetradecanyl-(1,4-phenylene-bis-(methylene)]-2-aminoethyl-2-pyridine or a pharmaceutically acceptable salt thereof.

17. The method of claim 1, wherein the compound of formula (1) is selected from the group consisting of:
3,3′-bis-1,5,9,13-tetraazacyclohexadecane;
3,3′-bis-1,5,8,11,14-pentaazacyclohexadecane;
5,5′-bis-1,4,8,11-tetraazacyclotetradecane;
2,5′-bis-1,4,8,11-tetraazacyclotetradecane;
2,6′-bis-1,4,8,11-tetraazacyclotetradecane;
methylene (or polymethylene) di 1-N-1,4,8,11-tetraazacyclotetradecane;
11,11′(1,2-ethanediyl)bis-1,4,8,11-tetraazacyclotetradecane;
11,11′-(1,2-propanediyl)bis-1,4,8,11-tetraazacyclotetradecane;
11,11′(1,2-butanediyl)bis-1,4,8,11-tetraazacyclotetradecane;
11,11′-(1,2-pentanediyl)bis-1,4,8,11-tetraazacyclotetradecane;
11,11′-(1,2-hexanediyl)bis-1,4,8,11-tetraazacyclotetradecane;
1,1′-[1,3-phenylene-bis(methylene)]-bis-1,4,8,11-tetraazacyclotetradecane;
1,1′-[1,4-phenylene-bis(methylene)]-bis-1,4,8,11-tetraazacyclotetradecane;
1,1′-[3,3′-biphenylene-bis-(methylene)]-bis-1,4,8,11-tetraazacyclotetradecane;
11,11′-[1,4-phenylene-bis-(methylene)]-bis-1,4,7,11-tetraazacyclotetradecane;
1,11′-[1,4-phenylene-bis(methylene)]-1,4,8,11-tetraazacyclotetradecane;
1,1′-[2,6-pyridine-bis-(methylene)]-bis-1,4,8,11-tetraazacyclotetradecane;
1,1-[3,5-pyridine-bis-(methylene)]-bis-1,4,8,11-tetraazacyclotetradecane;
1,1′-[2,5-thiophene-bis-(methylene)]-bis-1,4,8,11-tetraazacyclotetradecane;
1,1′-[4,4′-(2,2′-bipyridine)-bis-(methylene)]-bis-1,4,8,11-tetraazacyclotetradecane;
1,1′-[2,9-(1,10-phenanthroline)-bis-(methylene)]-bis-1,4,8,11-tetraazacyclotetradecane;
1,1′-[1,3-phenylene-bis-(methylene)]-bis-1,4,7,10-tetraazacyclotetradecane;
1,1′-[1,4-phenylene-bis-(methylene)]-bis-1,4,7,10-tetraazacyclotetradecane;
1,1′-[5-nitro-1,3-phenylenebis(methylene)]bis-1,4,8,11-tetraazacyclotetradecane;
1,1′-[2,4,5,6-tetrachloro-1,3-phenyleneis(methylene)]bis-1,4,8,11-tetraazacyclotetradecane;
1,1′-[2,3,5,6-tetrafluoro-1,4-phenylenebis(methylene)]bis-1,4,8,11-tetraazacyclotetradecane;
1,1′-[1,4-naphthylene-bis-(methylene)]bis-1,4,8,11-tetraazacyclotetradecane;
1,1′-[1,3-phenylenebis-(methylene)]bis-1,5,9-triazacyclododecane;
1,1′-[1,4-phenylene-bis-(methylene)]-1,5,9-triazacyclododecane;
1,1′-[2,5-dimethyl-1,4-phenylenebis-(methylene)]-bis-1,4,8,11-tetraazacyclotetradecane;
1,1′-[2,5-dichloro-1,4-phenylenebis-(methylene)]-bis-1,4,8,11-tetraazacyclotetradecane;
1,1′-[2-bromo-1,4-phenylenebis-(methylene)]-bis-1,4,8,11-tetraazacyclotetradecane;
1,1′-[6-phenyl-2,4-pyridinebis-(methylene)]-bis-1,4,8,11-tetraazacyclotetradecane;
7,7′-[1,4-phenylene-bis(methylene)]bis-3,7,11,17-tetraazabicyclo[13.3.1]heptadeca-1(17),13,15-triene;
7,7′-[1,4-phenylene-bis(methylene)]bis[15-chloro-3,7,11,17-tetraazabicyclo[13.3.1]heptadeca-1(17),13,15-triene];
7,7′-[1,4-phenylene-bis(methylene)]bis[15-methoxy-3,7,11,17-tetraazabicyclo[13.3.1]heptadeca-1(17),13,15-triene];
7,7′-[1,4-phenylene-bis(methylene)]bis-3,7,11,17-tetraazabicyclo[13.3.1]-heptadeca-13,16-triene-15-one;
7,7′-[1,4-phenylene-bis(methylene)]bis-4,7,10,17-tetraazabicyclo[13.3.1]-heptadeca-1(17),13,15-triene;
8,8′-[1,4-phenylene-bis(methylene)]bis-4,8,12,19-tetraazabicyclo[15.3.1]nonadeca-1(19),15,17-triene;
6,6′-[1,4-phenylene-bis(methylene)]bis-3,6,9,15-tetraazabicyclo[11.3.1]pentadeca-1(15),11,13-triene;
6,6′-[1,3-phenylene-bis(methylene)]bis-3,6,9,15-tetraazabicyclo[11.3.1]pentadeca-1(15),11,13-triene;
17,17′-[1,4-phenylene-bis(methylene)]bis-3,6,14,17,23,24-hexaazatricyclo[17.3.1.18,12]tetracosa-1(23),8,10,12(24),19,21-hexaene;
N-[1,4,8,11-tetraazacyclotetradecanyl-1,4-phenylenebis(methylene)]-2-(amino-methyl)pyridine;
N-[1,4,8,11-tetraazacyclotetradecanyl-1,4-phenylenebis(methylene)]-N-methyl-2-(aminomethyl)pyridine;
N-[1,4,8,11-tetraazacyclotetradecanyl-1,4-phenylenebis(methylene)]-4-(amino-methyl)pyridine;
N-[1,4,8,11-tetraazacyclotetradecanyl-1,4-phenylenebis(methylene)]-3-(amino-methyl)pyridine;
N-[1,4,8,11-tetraazacyclotetradecanyl-1,4-phenylenebis(methylene)]-(2-amino-methyl-5-methyl)pyrazine;
N-[1,4,8,11-tetraazacyclotetradecanyl-1,4-phenylenebis(methylene)]-2-(amino-ethyl)pyridine;
N-[1,4,8,11-tetraazacyclotetradecanyl-1,4-phenylenebis(methylene)]-2-(amino-methyl)thiophene;
N-[1,4,8,11-tetraazacyclotetradecanyl-1,4-phenylenebis(methylene)]-2-(amino-ethyl)mercaptan;
N-[1,4,8,11-tetraazacyclotetradecanyl-1,4-phenylenebis(methylene)]-2-amino-benzylamine;
N-[1,4,8,11-tetraazacyclotetradecanyl-1,4-phenylenebis(methylene)]-4-amino-benzylamine;
N-[1,4,8,11-tetraazacyclotetradecanyl-1,4-phenylenebis(methylene)]-4-(amino-ethyl)imidazole;
N-[1,4,8,11-tetraazacyclotetradecanyl-1,4-phenylenebis(methylene)]-benzylamine;
N-[1,4,8,11-tetraazacyclotetradecanyl-1,4-phenylenebis(methylene)]-purine;
N-[1,4,8,11-tetraazacyclotetradecanyl-1,4-phenylenebis(methylene)]-4-phenylpiperazine;
1-[2,6-dimethoxypyrid-4-yl(methylene)]-1,4,8,11-tetraazacyclotetradecane;
1-[2-chloropyrid-4-yl(methylene)]-1,4,8,11-tetraazacyclotetradecane;
1-[2,6-dimethylpyrid-4-yl(methylene)]-1,4,8,11-tetraazacyclotetradecane;
1-[2-methylpyrid-4-yl(methylene)]-1,4,8,11-tetraazacyclotetradecane;
1-[2,6-dichloropyrid-4-yl(methylene)]-1,4,8,11-tetraazacyclotetradecane;
1-[2-chloropyrid-5-yl(methylene)]-1,4,8,11-tetraazacyclotetradecane;
7-[4-methylphenyl(methylene)]-4,7,10,17-tetraazabicyclo[13.3.1]heptadeca-1(17),13,15-triene;
N-[4-(1,4,7-triazacyclotetra-decanyl)-1,4-phenylenebis(methylene)]-2-(aminomethyl)pyridine;
N-[1-(1,4,7-triazacyclotetra-decanyl)-1,4-phenylenebis(methylene)]-2-(aminomethyl)pyridine;
N-[7-(4,7,10,17-tetraazabicyclo[13.3.1]heptadeca-1(17),13,15-trienyl)-1,4-phenylenebis(methylene)]-2-(aminomethyl)pyridine;
N-[7-(4,7,10-triazabicyclo[13.3.1]heptadeca-1(17),13,15-trienyl)-1,4-phenylenebis(methylene)]-2-(aminomethyl)pyridine;
N-[4-[4,7,10-triazabicyclo[13.3.1]heptadeca-1(17),13,15-trienyl]-1,4-phenylenebis(methylene)]-2-(aminomethyl)pyridine;
N-[4-[4,7,10,17-tetraazabicyclo[13.3.1]heptadeca-1(17),13,15-trienyl]-1,4-phenylenebis(methylene)]-2-(aminomethyl)pyridine;
N-[3-(3,6,17-triazabicyclo[13.3.1]heptadeca-1(17),13,15-trienyl)-1,4-phenylenebis(methylene)]-2-(aminomethyl)pyridine;
N-[3-(3,6,17-triazabicyclo[13.3.1]heptadeca-1(17),13,15-trienyl)-1,3-phenylenebis(methylene)]-2-(aminomethyl)pyridine;
N-[4-(4,7,17-triazabicyclo[13.3.1]heptadeca-1(17),13,15-trienyl)-1,4-phenylenebis(methylene)]-2-(aminomethyl)pyridine;
N-[7-(4,7,17-triazabicyclo[13.3.1]heptadeca-1(17),13,15-trienyl)-1,4-phenylenebis(methylene)]-2-(aminomethyl)pyridine;
N-[6-(3,6,9-triazabicyclo[11.3.1]pentadeca-1(15),11,13-trienyl)-1,3-phenylenebis(methylene)]-2-(aminomethyl)pyridine;
N-[4-(1,7-diazacyclotetradecanyl)-1,4-phenylenebis(methylene)]-2-(aminomethyl)pyridine;
N-[7-(4,10-diazabicyclo[13.3.1]heptadeca-1(17),13,15-trienyl)-1,4-phenylenebis(methylene)]-2-(aminomethyl)pyridine;
N-[7-(4,10,17-triazabicyclo[13.3.1]heptadeca-1(17),13,15-trienyl)-1,4-phenylenebis(methylene)]-2-(aminomethyl)pyridine;
N-[4-(11-fluoro-1,4,7-triazacyclotetradecanyl)-1,4-phenylenebis(methylene)]-2-(aminomethyl)pyridine;
N-[4-(11,11-difluoro-1,4,7-triazacyclotetradecanyl)-1,4-phenylenebis(methylene)]-2-(aminomethyl)pyridine;
N-[4-(1,4,7-triazacyclotetradecan-2-onyl)-1,4-phenylenebis(methylene)]-2-(aminomethyl)pyridine;
N-[12-(5-oxa-1,9-diazacyclotetradecanyl)-1,4-phenylenebis(methylene)]-2-(aminomethyl)pyridine;
N-[4-(11-oxa-1,4,7-triazacyclotetradecanyl)-1,4-phenylenebis(methylene)]-2-(aminomethyl)pyridine;
N-[4-(11-thia-1,4,7-triazacyclotetradecanyl)-1,4-phenylenebis(methylene)]-2-(aminomethyl)pyridine;
N-[4-(11-sulfoxo-1,4,7-triazacyclotetradecanyl)-1,4-phenylenebis(methylene)]-2-(aminomethyl)pyridine;
N-[4-(11-sulfono-1,4,7-triazacyclotetradecanyl)-1,4-phenylenebis(methylene)]-2-(aminomethyl)pyridine; and
N-[4-(3-carboxo-1,4,7-triazacyclotetradecanyl)-1,4-phenylenebis(methylene)]-2-(aminomethyl)pyridine;
or a pharmaceutically acceptable salt thereof.

18. The method of claim 1, wherein the immunotherapeutic agent is selected from the group consisting of a human monoclonal antibody, a mouse monoclonal antibody, a chimeric monoclonal antibody, a humanized monoclonal antibody and a combination thereof.

19. The method of claim 1, wherein the immunotherapeutic agent is selected from the group consisting of an unconjugated antibody, a radiolabeled antibody, a chemolabeled antibody, an immunotoxin and a combination thereof.

20. The method of claim 1, wherein the immunotherapeutic agent is selected from the group consisting of alemtuzumab, rituximab, gemtuzumab ozogamicin, ibritumomab tiuxetan, tositumomab and a combination thereof.

21. The method of claim 20, wherein the compound of formula (1) is selected from the group consisting of
1,1′-[1,4-phenylene-bis-(methylene)-bis-1,4,8,11-tetraazacyclotetradecane or a pharmaceutically acceptable salt thereof and N-[1,4,8,11-tetraazacyclotetradecanyl-(1,4-phenylene-bis-(methylene)]-2-aminoethyl-2-pyridineor or a pharmaceutically acceptable salt thereof.

22. The method of claim 1, wherein the hematological malignancy is selected from the group consisting of acute lymphoblastic leukemia, acute myelogenous leukemia, chronic myelogenous leukemia, chronic lymphocytic leukemia, hairy cell leukemia, Hodgkin's lymphoma, non-Hodgkin's lymphoma, multiple myeloma and a combination thereof.

23. The method of claim 1, wherein the medicament is administered in combination with granulocyte-colony stimulating factor (G-CSF).

24. A pharmaceutical composition for treating a hematological malignancy, said composition comprising a therapeutically effective amount of a CXCR4 antagonist or a pharmaceutically acceptable salt or prodrug thereof and a therapeutically effective amount of an immunotherapeutic agent in unit dosage form.

25. The pharmaceutical composition of claim 24, wherein the CXCR4 antagonist is a compound of the formula
Z-linker-Z′ (1)
or a pharmaceutically acceptable salt or prodrug thereof,
wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;
Z′ may be embodied in a form as defined by Z above, or alternatively may be of the formula
—N(R)—(CR₂)ₙ—X
wherein each R is independently H or straight, branched or cyclic alkyl (1-6C), n is 1 or 2, and X is an aromatic ring, including heteroaromatic rings, or is a mercaptan,
or Z′ may be of the formula
—Ar(Y)ⱼ
wherein Ar is an aromatic or heteroaromatic moiety, and each Y is independently a non-interfering substituent and j is 0-3; and
“linker” represents a bond, alkylene (1-6C) or may comprise aryl, fused aryl, oxygen atoms contained in an alkylene chain, or may contain keto groups or nitrogen or sulfur atoms.

26. The pharmaceutical composition of claim 25, wherein the compound of formula (1) is selected from the group consisting of:
3,3′-bis-1,5,9,13-tetraazacyclohexadecane;
3,3′-bis-1,5,8,11,14-pentaazacyclohexadecane;
5,5′-bis-1,4,8,11-tetraazacyclotetradecane;
2,5′-bis-1,4,8,11-tetraazacyclotetradecane;
2,6′-bis-1,4,8,11-tetraazacyclotetradecane;
methylene (or polymethylene) di 1-N-1,4,8,11-tetraazacyclotetradecane;
11,11′(1,2-ethanediyl)bis-1,4,8,11-tetraazacyclotetradecane;
11,11′(1,2-propanediyl)bis-1,4,8,11-tetraazacyclotetradecane;
11,11′(1,2-butanediyl)bis-1,4,8,11-tetraazacyclotetradecane;
11,11′(1,2-pentanediyl)bis-1,4,8,11-tetraazacyclotetradecane;
11,11′-(1,2-hexanediyl)bis-1,4,8,11-tetraazacyclotetradecane;
1,1′-[1,3-phenylene-bis(methylene)]-bis-1,4,8,11-tetraazacyclotetradecane;
1,1′-[1,4-phenylene-bis(methylene)]-bis-1,4,8,11-tetraazacyclotetradecane;
1,1′-[3,3′-biphenylene-bis-(methylene)]-bis-1,4,8,11-tetraazacyclotetradecane;
11,11′-[1,4-phenylene-bis-(methylene)]-bis-1,4,7,11-tetraazacyclotetradecane;
1,11′-[1,4-phenylene-bis(methylene)]-1,4,8,11-tetraazacyclotetradecane;
1,1′-[2,6-pyridine-bis-(methylene)]-bis-1,4,8,11-tetraazacyclotetradecane;
1,1-[3,5-pyridine-bis-(methylene)]-bis-1,4,8,11-tetraazacyclotetradecane;
1,1′-[2,5-thiophene-bis-(methylene)]-bis-1,4,8,11-tetraazacyclotetradecane;
1,1′-[4,4′-(2,2′-bipyridine)-bis-(methylene)]-bis-1,4,8,11-tetraazacyclotetradecane;
1,1′-[2,9-(1,10-phenanthroline)-bis-(methylene)]-bis-1,4,8,11-tetraazacyclotetradecane;
1,1′-[1,3-phenylene-bis-(methylene)]-bis-1,4,7,10-tetraazacyclotetradecane;
1,1′-[1,4-phenylene-bis-(methylene)]-bis-1,4,7,10-tetraazacyclotetradecane;
1,1′-[5-nitro-1,3-phenylenebis(methylene)]bis-1,4,8,11-tetraazacyclotetradecane;
1,1′-[2,4,5,6-tetrachloro-1,3-phenyleneis(methylene)]bis-1,4,8,11-tetraazacyclotetradecane;
1,1′-[2,3,5,6-tetrafluoro-1,4-phenylenebis(methylene)]bis-1,4,8,11-tetraazacyclotetradecane;
1,1′-[1,4-naphthylene-bis-(methylene)]bis-1,4,8,11-tetraazacyclotetradecane;
1,1′-[1,3-phenylenebis-(methylene)]bis-1,5,9-triazacyclododecane;
1,1′-[1,4-phenylene-bis-(methylene)]-1,5,9-triazacyclododecane;
1,1′-[2,5-dimethyl-1,4-phenylenebis-(methylene)]-bis-1,4,8,11-tetraazacyclotetradecane;
1,1′-[2,5-dichloro-1,4-phenylenebis-(methylene)]-bis-1,4,8,11-tetraazacyclotetradecane;
1,1′-[2-bromo-1,4-phenylenebis-(methylene)]-bis-1,4,8,11-tetraazacyclotetradecane;
1,1′-[6-phenyl-2,4-pyridinebis-(methylene)]-bis-1,4,8,11-tetraazacyclotetradecane;
7,7′-[1,4-phenylene-bis(methylene)]bis-3,7,11,17-tetraazabicyclo[13.3.1]heptadeca-1(17),13,15-triene;
7,7′-[1,4-phenylene-bis(methylene)]bis[15-chloro-3,7,11,17-tetraazabicyclo[13.3.1]heptadeca-1(17),13,15-triene];
7,7′-[1,4-phenylene-bis(methylene)]bis[15-methoxy-3,7,11,17-tetraazabicyclo[13.3.1]heptadeca-1(17),13,15-triene];
7,7′-[1,4-phenylene-bis(methylene)]bis-3,7,11,17-tetraazabicyclo[13.3.1]-heptadeca-13,16-triene-15-one;
7,7′-[1,4-phenylene-bis(methylene)]bis-4,7,10,17-tetraazabicyclo[13.3.1]-heptadeca-1(17),13,15-triene;
8,8′-[1,4-phenylene-bis(methylene)]bis-4,8,12,19-tetraazabicyclo[15.3.1]nonadeca-1(19),15,17-triene;
6,6′-[1,4-phenylene-bis(methylene)]bis-3,6,9,15-tetraazabicyclo[11.3.1]pentadeca-1(15),11,13-triene;
6,6′-[1,3-phenylene-bis(methylene)]bis-3,6,9,15-tetraazabicyclo[11.3.1]pentadeca-1(15),11,13-triene;
17,17′-[1,4-phenylene-bis(methylene)]bis-3,6,14,17,23,24-hexaazatricyclo[17.3.1.18,12]tetracosa-1(23),8,10,12(24),19,21-hexaene;
N-[1,4,8,11-tetraazacyclotetradecanyl-1,4-phenylenebis(methylene)]-2-(amino-methyl)pyridine;
N-[1,4,8,11-tetraazacyclotetradecanyl-1,4-phenylenebis(methylene)]-N-methyl-2-(aminomethyl)pyridine;
N-[1,4,8,11-tetraazacyclotetradecanyl-1,4-phenylenebis(methylene)]-4-(amino-methyl)pyridine;
N-[1,4,8,11-tetraazacyclotetradecanyl-1,4-phenylenebis(methylene)]-3-(amino-methyl)pyridine;
N-[1,4,8,11-tetraazacyclotetradecanyl-1,4-phenylenebis(methylene)]-(2-amino-methyl-5-methyl)pyrazine;
N-[1,4,8,11-tetraazacyclotetradecanyl-1,4-phenylenebis(methylene)]-2-(amino-ethyl)pyridine;
N-[1,4,8,11-tetraazacyclotetradecanyl-1,4-phenylenebis(methylene)]-2-(amino-methyl)thiophene;
N-[1,4,8,11-tetraazacyclotetradecanyl-1,4-phenylenebis(methylene)]-2-(amino-ethyl)mercaptan;
N-[1,4,8,11-tetraazacyclotetradecanyl-1,4-phenylenebis(methylene)]-2-amino-benzylamine;
N-[1,4,8,11-tetraazacyclotetradecanyl-1,4-phenylenebis(methylene)]-4-amino-benzylamine;
N-[1,4,8,11-tetraazacyclotetradecanyl-1,4-phenylenebis(methylene)]-4-(amino-ethyl)imidazole;
N-[1,4,8,11-tetraazacyclotetradecanyl-1,4-phenylenebis(methylene)]-benzylamine;
N-[1,4,8,11-tetraazacyclotetradecanyl-1,4-phenylenebis(methylene)]-purine;
N-[1,4,8,11-tetraazacyclotetradecanyl-1,4-phenylenebis(methylene)]-4-phenylpiperazine;
1-[2,6-dimethoxypyrid-4-yl(methylene)]-1,4,8,11-tetraazacyclotetradecane;
1-[2-chloropyrid-4-yl(methylene)]-1,4,8,11-tetraazacyclotetradecane;
1-[2,6-dimethylpyrid-4-yl(methylene)]-1,4,8,11-tetraazacyclotetradecane;
1-[2-methylpyrid-4-yl(methylene)]-1,4,8,11-tetraazacyclotetradecane;
1-[2,6-dichloropyrid-4-yl(methylene)]-1,4,8,11-tetraazacyclotetradecane;
1-[2-chloropyrid-5-yl(methylene)]-1,4,8,11-tetraazacyclotetradecane;
7-[4-methylphenyl(methylene)]-4,7,10,17-tetraazabicyclo[13.3.1]heptadeca-1(17),13,15-triene;
N-[4-(1,4,7-triazacyclotetra-decanyl)-1,4-phenylenebis(methylene)]-2-(aminomethyl)pyridine;
N-[1-(1,4,7-triazacyclotetra-decanyl)-1,4-phenylenebis(methylene)]-2-(aminomethyl)pyridine;
N-[7-(4,7,10,17-tetraazabicyclo[13.3.1]heptadeca-1(17),13,15-trienyl)-1,4-phenylenebis(methylene)]-2-(aminomethyl)pyridine;
N-[7-(4,7,10-triazabicyclo[13.3.1]heptadeca-1(17),13,15-trienyl)-1,4-phenylenebis(methylene)]-2-(aminomethyl)pyridine;
N-[4-[4,7,10-triazabicyclo[13.3.1]heptadeca-1(17),13,15-trienyl]-1,4-phenylenebis(methylene)]-2-(aminomethyl)pyridine;
N-[4-[4,7,10,17-tetraazabicyclo[13.3.1]heptadeca-1(17),13,15-trienyl]-1,4-phenylenebis(methylene)]-2-(aminomethyl)pyridine;
N-[3-(3,6,17-triazabicyclo[13.3.1]heptadeca-1(17),13,15-trienyl)-1,4-phenylenebis(methylene)]-2-(aminomethyl)pyridine;
N-[3-(3,6,17-triazabicyclo[13.3.1]heptadeca-1(17),13,15-trienyl)-1,3-phenylenebis(methylene)]-2-(aminomethyl)pyridine;
N-[4-(4,7,17-triazabicyclo[13.3.1]heptadeca-1(17),13,15-trienyl)-1,4-phenylenebis(methylene)]-2-(aminomethyl)pyridine;
N-[7-(4,7,17-triazabicyclo[13.3.1]heptadeca-1(17),13,15-trienyl)-1,4-phenylenebis(methylene)]-2-(aminomethyl)pyridine;
N-[6-(3,6,9-triazabicyclo[11.3.1]pentadeca-1(15),11,13-trienyl)-1,3-phenylenebis(methylene)]-2-(aminomethyl)pyridine;
N-[4-(1,7-diazacyclotetradecanyl)-1,4-phenylenebis(methylene)]-2-(aminomethyl)pyridine;
N-[7-(4,10-diazabicyclo[13.3.1]heptadeca-1(17),13,15-trienyl)-1,4-phenylenebis(methylene)]-2-(aminomethyl)pyridine;
N-[7-(4,10,17-triazabicyclo[13.3.1]heptadeca-1(17),13,15-trienyl)-1,4-phenylenebis(methylene)]-2-(aminomethyl)pyridine;
N-[4-(11-fluoro-1,4,7-triazacyclotetradecanyl)-1,4-phenylenebis(methylene)]-2-(aminomethyl)pyridine;
N-[4-(11,11-difluoro-1,4,7-triazacyclotetradecanyl)-1,4-phenylenebis(methylene)]-2-(aminomethyl)pyridine;
N-[4-(1,4,7-triazacyclotetradecan-2-onyl)-1,4-phenylenebis(methylene)]-2-(aminomethyl)pyridine;
N-[12-(5-oxa-1,9-diazacyclotetradecanyl)-1,4-phenylenebis(methylene)]-2-(aminomethyl)pyridine;
N-[4-(11-oxa-1,4,7-triazacyclotetradecanyl)-1,4-phenylenebis(methylene)]-2-(aminomethyl)pyridine;
N-[4-(11-thia-1,4,7-triazacyclotetradecanyl)-1,4-phenylenebis(methylene)]-2-(aminomethyl)pyridine;
N-[4-(11-sulfoxo-1,4,7-triazacyclotetradecanyl)-1,4-phenylenebis(methylene)]-2-(aminomethyl)pyridine;
N-[4-(11-sulfono-1,4,7-triazacyclotetradecanyl)-1,4-phenylenebis(methylene)]-2-(aminomethyl)pyridine; and
N-[4-(3-carboxo-1,4,7-triazacyclotetradecanyl)-1,4-phenylenebis(methylene)]-2-(aminomethyl)pyridine;
or a pharmaceutically acceptable salt thereof.

27. The pharmaceutical composition of claim 24, wherein the immunotherapeutic agent is selected from the group consisting of a human monoclonal antibody, a mouse monoclonal antibody, a chimeric monoclonal antibody, a humanized monoclonal antibody, and a combination thereof.

28. The pharmaceutical composition of claim 24, wherein the immunotherapeutic agent is selected from the group consisting of an unconjugated antibody, a radiolabeled antibody, a chemolabeled antibody, an immunotoxin and a combination thereof.

29. The pharmaceutical composition of claim 24, wherein the immunotherapeutic agent is selected from the group consisting of alemtuzumab, rituximab, gemtuzumab ozogamicin, ibritumomab tiuxetan, tositumomab and a combination thereof.

30. The pharmaceutical composition of claim 29, wherein the compound of formula (1) is selected from
1,1′-[1,4-phenylene-bis-(methylene)-bis-1,4,8,11-tetraazacyclotetradecane or a pharmaceutically acceptable salt thereof and N-[1,4,8,11-tetraazacyclotetradecanyl-(1,4-phenylene-bis-(methylene)]-2-aminoethyl-2-pyridine or a pharmaceutically acceptable salt thereof.

31-53. (canceled)

54. The pharmaceutical composition of claim 24, wherein the CXCR4 antagonist is present as a low dose.

55. The pharmaceutical composition claim 54, wherein the low dose of the CXCR4 antagonist is selected from the group consisting of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg and 1 mg/kg.

56. The pharmaceutical composition of claim 24, wherein the hematological malignancy is selected from the group consisting of acute lymphoblastic leukemia, acute myelogenous leukemia, chronic myelogenous leukemia, chronic lymphocytic leukemia, hairy cell leukemia, Hodgkin's lymphoma, non-Hodgkin's lymphoma, multiple myeloma and a combination thereof.
